SEQUENCE LISTING (24 sequences)

SEQ ID NO: 1 - DNA, 12738 bp, Lotus japonicus
  promoter (1)..(2359): promoter + 5′ UTR of transcript
cccgggattgttcatatccccatgtgtgtgtttggtttggacacaagcaagcttgccacagtggtgtttacaactcattctgaattatatatatatatatatatatataactaatctctatcaataatttagttagttttatgggataatatgattcccgtcaaattagcttgtaggttaaaaatccatatttatataataaattagacttatctaaatcacttaagtaaatatgatgtaaaatcacaatagcttaggtagtttaacaatgttttatcatgaatttaaaaaaaattagttgtttcaaaatattattggaccaaataagcaaagaggagctaatccaattataaacagcacatctttataattgttttctactcgatctaatattggttccttctttccttgagaaaaaaagggttaaaaatgcgatttgttttgtaaaattaatttgtgacattaacaagagaaaactctacttttttaatttttaagaagttacttaaatatagaaagaaagaagttacttaaatatgaaacttgtctaaccaaagtcaaccgatcaaagcgtttggttagcaaagagaacttaaaaaaataactaaattaactgtttgaacagtgatgtaacagagaaatagaatggcttacatatggattatggagttaatggaaggcacactagttttagttcacggcaacaagacagaattatcctcctatattttaggaggttttcttatttatttttaatgtaagacgtgtatccttcttttgaggtaataatgttcgagcttgaaacattcacattatagtgtgactaattgcttaatatgaatcaagcatgtaccacttgaaacactcactcatcgtttataatataattactttatagtactataaagtatgtaattattttctcctttttaagtatctcaatcatttttttgacccaaatattattttagagaagaaacatgtcttaagaccaatacatcaacatatatggagagtagtttgacaacctctcatgagctttctaaagccaaacaaatgtactcacataaaaaaattaaaaatttacagcaagtgagttataaagagtgattaataaatgttgaataacgatggagggtagttatgaaattccataaataaagcaaaggatgtttatggcaattgacatgggaataagatccgcgcgttgtcgccgtgtcataatcgctcagatttgtgatagcgagagatttccattcttttcctcttttttcgaacgaacgaacgaacgaacacagtagcagctgtattaggattcagattgcatgatacattgatattgatattgatattgatattgatattgttgtgggtcaagtctctactctactatccaaagtaagcatatatatagagagagagagagagcttgggactagggaggatattagcttatgtgactgtgaagttgaagggagacaagagcgtatctggcaaaatcctcaaaataaaatactagtactagtacagagaaaagagactaagagagagagtgctgctgcacatcaagacccattgtgatttgtgatttgtgatttgtgatttgtgatttgtgagtgagttcattgtacaggtattattgtttgttgtttctctcctcaaccaccctctaaagtctaatctaactcattgggctctgtgcttagctggttgtgttttgtgtatggtgaattagggggtcaatctctggttttcatcattattatattatatatgggaataccgtgctctcttctttgtccttcaccaaaactagtttcacgcctacacaacatgattagagcctcttcatttttttaatctcatcctttaagtgtatttctatttctattggctattgggacaaggggaaggtggtgcttcttaggaacttgagctgttttccatcttttgagacccatgctttgtctctctcatttttaattctgggtctctttcttctcttgtcctgattttttaaatgtgcttcttttttgcttcttacaaccaccctctaaaccattcatcatgcttggtttgcttttgcttctcctttcacaggtttcaatcacgcaaaacaatgctgcaatgatgctgtacttggagtttcttctgtgaccccttttttccttccttcaacaatcaacccaccagagaaaagtgtctcagattttgagactactttcaactttcaaaacaaagtggatgggatcttcatcttatataaccacacatcaatcatttgtgctacttctccaattttctttagagatgaaatgaagagctaagcagacaagacaagtttatttgtttgttgctgatgggtcttgggttcaagatgcagcagagccaccaccctgtggctttgaagttacatgagcaagctgggagccagagaaagttcactttcattcagaacttcagaaactggtttctaccccttctgtttgtatggttcattgttatggctgcatttggtgcctgcatctaccataaaatggatgctgaaactaaagtcagaaggaaagaggtgctgggtagcctctgtgatcaaagggctagaatgctacaagaccaattcagtgtcagtgtcaaccatgtccatgcccttgccatccttgtttcaaccttccattactacagaaatacttcagccattgaccaggtttgtgcttgattttcctttccttgaagcattttttagttggaggctcaatttctttttctgatttgattctggccttaaaaattagaatcaattgtagaaggatttccaaacatgcccattttggaaattggtgcatctgatagtatcatgtttagatcagtttctttttcctcagaattgattttgggcttaaaatcaattgtggaaggatattcattagtaatttggatattgttgcatcatatggttctatctagttacatcatttttttccactctgattgcatgtatctttctcctgttcttttccctatcaggaaacctttgcagaatacacggccaggacagcatttgaacggccattaatgagtggggtggcctatgcacagagagtggttcactcagagagagaaagatttgagaagcaacatgggtgggttataaagacaatggaaagagtgccttcaggggttagggatgagtatgcagcagtgatatttgcacaggaaactgtctcttaccttgaatctattgatatgatgtctggggaggtaaatgtcaacacttgtgaattaattgtaaaactcagaagctactcagagaagctcttccccagaattggttctgcctttagaataaattgtacatggatttgaccacattttctcatttgcatgatgcaggaggaccgagagaacattttgagggctagagccactgggaaagctgttctgactagccctttcagactgctggattctcatcatcttggcgtggttctaacatttcctgtttataaatctaagctccctccagagccaacgacggaagaggtcattaaagccatagcagggtatgtcctcatttcacttttcttgccaaaaccagacttctatttggttgtgtttccgtaggctatgactgatatgtagtttcaactcagttagactataatataaacccttcatgtggcccttacccaacagcttaagcttttgggataattggttgcttgacaaactccttccgtagaaaacttggttagctttggttctatgtgggctttatgttttccctgagcttatgtaatagcatgatgtgtttaatgtactttttaatggaaacagatatattggaggatcctttgatgttgagtcccttgtggagaatttacttggtcaacttgctggtaaccaagcaattttggtgaaggtatatgatataacaaactctagcgaccccctaatcatgtatggcagccaatatgaagagggtgatatgtctcttgtccatgaaagtaagcttgattttggagatccatacaggaaacatcacatgatctgtaggtgggtgcttctagttattgttgtagtacattttctgagtttcagtggtttatcaattatcagcagattcttatgatcaatttttttaacagatatcaccaacaggcaccaacaaattggatagcatataccacggcattcctattctttgtgattctttgtttagtgggttacattttatatgctgctggaactcacattgtcaaggtagaagatgattacaatgcaatgcaggatttaaaagtcaaagcagaagcagctgatattgccaagtcacaggtacttttcatgacatgttagcactgttcgttatttccttgaattgcatactgatcactagaaactgaaaatttgttattaatgtcagtttctagctaccgtctctcatgaaattagaactcccatgaatggaattttaggtaactttaagattctctctcgttctctttccactgaaaagcaacacatgctttcattccatacctgatactttcccattagtgatgctatcgttaaactccttgtcactgtaggaatgcttggtctgcttttacgcacagaattgagttcaacacaaagagactatgctcagactgctcaagcatgtgggaaggcactaatagcattaataaatgaggtgcttgaccgagctaaaattgaagcaggcaaattagagctagaagcagttccatttgaccttcgttccatacttgacgatgtcctttctcttttttctgagaagtcaagacacaaaggcttagaggtacgtttagtcattgctaaatctgttgtgaagtcctgtacaagtggcgtaatttcatagtcctatttccttttcttcaattgatgatcttatttcatattcctctcgtgtttctctctttctatgttgccatgtcgttgggcggtgatgttcctactctgatcccaaattcctgatgtgcaattattccgacttggacttcaatggtttgggaagtatagctggcagtgtttgtttctgacaaagttccggatatagttatgggcgatcctgggcgattcagacaaatagtgacaaatcttgttggaaactctgttaaggttagtggaattttcaaactttatttgcctaatgttgtgtgcaagttgtgtgttggaaatgcgtccttttaacgttataaaatcgtacaagttcgtattctccattgtatacaataacttattagcaaagtacttgttgatatcattactgattaactttaatatcttgcagttcactgagcgaggtcatatatttgttaaagtccatttagctgaaaaaagacagtgcacaatgaatggaaaatgtgagacttttctaaatggaggctgtgatgatgttttgcatgtatctggcagttataatttgaaaacccttagtggatatgaagccgctgatgaacggaacagctgggataattttaagcatcatattgctgacgaagaatttttctttgatgcttcggttaaaaagttggcctctagtgaatcttatgagcaagtcaccttgatggtcagcgtggaggacactggaattgggatttctttctctgcccaagatagtattttcatgccttttgtgcaggctgacagctcaacctctcgaaactatgggggtaccgggatcggcttgagtatcagtaagtgcttggttgaactgatgggcggtcagataaacttcataagccgaccccaggtcgggagcacgttttcattcactgcagatttcggaacatttaagaaaaactcaacaactgacatgaagaaacttaactttgaagatctaccttctagttttagaggtcttaaagccattgtggttgatggaaaacctgttagagctgcagtgactagataccatttgaagagactagggatacaagctaaagttgcaattagcatcaataaggctgtttctttatgtgggaaaaatggttctttgacctcggcgtaagtctttaattaacctttttggtttcaattatgtagaaatgtattgaatgttatgataatcagtagcattatcaacttttagtaattgttcttaacatatgctaatagtcatatctttctataatactacaatactgtagccatataatatctttcctgtattggagtgagttttcaaatgtttttctgtgatattttggaagttatcttcagtttgagaactcatttgtcatttttgcattttgttattggatatttggatggatctttacaaaggatgtgtggattttgacttgttatacacatttcttctccattttatattgtttgtgttattctttttactcataaagaaatttagaaactgcattgactggttctttttaattacttacagatattgacattgatattttttgtaaatgctgtcttgacatggtttaattacttacagactaggtttttctttccttttctaacatgcatatccatttacttttttgaccaaccaacatcctcatgagtcatgacatgttgatgatttatatggttgacttgagactatttagacattaaataaccgcaaattccatgttgtttgtgtgtttggttccctgttgggtaatctcagaatcaattatgatagagtaaaatcaattttggatgagatgtgtgggtgtcattttgtaaacctaaacccaaaatcaattctgctataagctagagagagtagttgaacataatcaattgtgagattttgcaagtggattgcacaacattgccctatgaaaatcactttttgttcacaaaatttatctaaacatacataacttcattttcaacctttactataatcaattttacaataattaattttacccaaaatcaattgtgacaatgagtttccaaacacacacttaaagactaccatttgcagaaaatatgtgatagaagacttatgtttatgtagtgtgtttcagttcattcactgatttaaactactcggattttgcagattatttcagcctgatattatttttgttgagaaggactcttgggtttctggagaggatggtggtatcttcaatgcgtttaagatgcctcaaatgatccttcttgcaaccaatatctgtaacgctgaatttgataaagccaaagctgcaggtttcagtgatacagtgatcatgaagccactgagagctagtatgctggctgcttgtcttcagcaagttttcgggactggcaagacgaggcagtttgggaaagacatgtcgaatggttcttcagtacgaagccttctttgcggaaagaaaatcttagtggttgatgataatttggtgaaccgaagggtcgccgccggcgcgttgaaaaactttggagctgatgtcaaatgtgcagcaagtggcaaagctgctcttgaaatgcttcaatatcctcacgatttcgatgcttgcttcatggatattcaaatgccagaaatggatgggtatgcttactggcactgactaatacatgttttttgccaacttaatatattactctttcaatattcgttgtgttattagaagatcatatagattaatttataaattttcttttagcaaaaccttatcaattaagtgtgtagaaaagtcagtctcacattatggtcaaataagtgttagggcaagcttcacctcaaagctagctatttgggtagatttaggcctaacccgaattctaagatggtatcagagtctatcctagatctttttattggaaaccacccgtatatgagcaactcgtagatattcattcttgaaagttgcacgctccatatgtccattcctaggtgcgagagagaagtctcactttgactagagatatgattaaaaaaatatttataaagggttgagcaatcctcacctcagagctaagcttttggggtaaagttaggcctaactcgaactctaataaagtgtttagctggtgtgtcaactgtcaatatgaaatcttttgcaatttactatgcattcacttacctactttattgaagcttattgacaatttgtgcagaagcatcattaattaggaacatgttagctatacaagttatgatgtttttgtatagcatatcatgttccaaccttccaataacaaaatatgtggttcaagtgtgagaatatataggttaaacaataaagtattgagttaacagaaatctaaacacacgctgtcactagctcttcatattgagacatgcatgggatttgacaaaacatctgaataaatatttgcaggtttgaggccactcgtcgaattcggatgatggaaagagaggcaagtgagcagctgaaaagtgaatctggtgaagaaaatggtaagaaaagtgagttccacatgcctatattggccatgacagctgatgtaatccatgctacatatgataagtgcttaaattgtgggatggatggatacgtctcaaagccttttgaagaagagaatctctatcaagcagttgcaaagttcttcaagtccaagcctgcttcagactcatgacactgcttattctgcagaacaggtcaaccaacttttgattgagaaacatttagtgttagcatgtttggatcaacttctcccagcatcaattctgaaactcagaagctactcatagaagcttctagccagaattgattttggcttcagaagctactaatggttttatgtagagagcaaaagttggtttccaagatatgcgaggatccatgatgatacacacgtctgaagtgatcatttctaaccagttgaagtttcactcgacgtgatttgaatccaagtaaatgcataccacataattatccatccccatcttgtgtacagattctcccaagggataagaaaatttatgtaaattcaatttttttcttttgcatctcaatacttccctgttagaactttttccctatgattatttccacttttcattttcaattatattttttgtaaaattggtctccatcctataaggtttgcttagttttttttaattcagatgagaaggttggtgcttataatgtgtatacctttttagcagtacttgtgatataaatatttgtcttatttttagaacttaggagataataagatcgtaggagtaaatagcatttttggagaatgatatcataattataatcatctacattctgcagttataaaaaaaatcaatattaatgcatagcaagtagctttaagaagtatgatctatttaagtattgaattttttttctaaactaattacaatgtattattgctgggatatgcgctttttactcgatagatatcatgtttggattagtttttcttagaatccattttggaaccgaaaaaattatcacataatttattcttcaaagttgattttggcaatcaaggtaaacactcataaaagctaaagggagttgtttcttattattgtgattttagctcattgtggtttttcaccgtgtatataaacatgcatttgatgtgacgttgacttgtgaagcacgtggctatccaccagcaatctcaagtaacacttgtcagcactatgttgctattataagtgacaaggcagtgaccacacccctctatatatgcataaacttttttattaaaagtaacagaatcaaaagggaagttttctacaaattaattaatggaaaattatcagaaccagtatggagctgttcctgctagcagggctccggttcagagccagagtgaaatattggaaaccaccaatgttggcggtgctacacaagtcataagaaatgattatgatggaagaaccactgctgcagatgctactgttgttggtgagagcgatctgcagcatcaccacaagaaaggaatattagagaagatcaaggagaagcttcctggaacacaccaccatcaagatcacaaatagctagctagctttgtttagtttattttgcttctatcattataaatgtaataataatagtatgtgtgttgctacatgcatgtgtatgtatcgtttagtcagctagtttgttaataacttgctttcaattttgcttccttacaagcctatttatgcaatgtatttggtcatttcactattggtttctaatcaatctaaattagtacatggtcctcttccttttagtgccttttaattaaagagtaaatgttgaatattgtttttagtcattctatttacatttttattttttttgcttcatcggaaaattatttacatttttattaagtgtgtgtttagattttcgtttttcgttaaaaatttcataattaattttttataacagtgatgtatgcagctaaggcatcctaacagacctatctttgtatgtatggcattctgggcgggtcggatttatttttagccccataattccccactactatacataaaatgatacctgctagtaaaataatttaaagtgtttcgaagtcatattggtaaattgatttcagttaaaaatcgaagtttgttagctactaaagacggtagcttctatgtcaaaatagttaattcctatattttataattggatcattttttctaaaatttattcaaacatctcaactcactttgactataatcactttataattaattttgttgaattaaataaattatattcaaattaaatgtgataatacccactgaatgtgattggaatgccatcagggaaatatggaacaaagagagctgcatgtggaagggcttgttatgtctgcaaaacagacaaagaagttgtaatcacaattaggacaattgccaaagatgtggggtcacattgacttgtcagggaaagtctatagatccactgtgctcttaaagctagaacaattaacataaaatttctgccaattaaggctttcacttcctatccctatctgctgtatattttaagtttgagtaaagggacagttgtggtgatgttataatcaactgcaggctgagggaaaagttgggggtcaagtggtgtctatataaacttttttgtcattattaatctttaatctttttggttttgtaaaatgacagcttttgtcttgtgctgaaagttgactgtgctagctagtaaagtcatgctggtttcttaattgattgtggtgtttttatgcgacaactgtagacattgcaatgcaggcacacaagtcaagaggcaattacaaacaaaatacagctattaataattgacctactaactactaaggagggaatttaaatgtgattttgaaatcttatattggatgtaaagaggcaatactcatcagactgtaaagtgacagaacaagtagatggtaaacaagtagaagtagaagcagcaacaaattgagtaaagaccatcaatgtcaacacttccttggagaagaatttcataagtgtcatgtgaaaataactagacaaaatgtagccaaaatgaagtatgaaactcagttattgaaacgagtgacactgactagattccaagttattgaaggaaccagcaaaaggttaagtgacagtgaagtgtgtgggttgaaagaggattgaataaaagctgaaccaatggactgaataggcaaacttgacaatttcccattagaatctgttttaacttccttgtacaaaagtttcttcggtgaaatttccagcattgttggtaacatgatgagtagctcatgaatctggaaaccaaacagttttgtttgttcaagctgtagagaagaatgagaactacatgtgagcattgttatgtaccaaaaaaacctgtgagcattgttgctgcaatcctggatcagtacgcatagcctgagcacaaagatgttgaattgaagtagttgctctaatatgttatgaatattcaagattaaaacatttttaagttatcatgtgacaatcacataactattaaattattttaatcttaactattaatcattagatctaacgatcatattctcatataaaataattattctgatacaataatatatatatatatatatatgtatgtaggggatctgaggatgtatcatatgagaaatgtgatattatgtgagaacatgagaataaatcacaaccgttagatttaatcaacggaccagattaaaacaatttaatgctcacatgaccacttaaaaatgtcatgtgactttcttaaaaagtcaaatgacttcatgttttaattttgaccgttcatgattagatttaacggtcatgatttattctcatgttctcacataaaaacatttttcatttttctgatatgtatatttgtaacataaaacacaacataaaatactatgtaaatcagccaatttcaccaaatacaatatgatgtgaaaaatataatattactattgatattcactcaaatgcacaatcggccctaaaaaatggtcaatttttctggttaatatatttcttttttaaaacatgagtattcatgcaatatatgatttcaaaatatataatttctttcaacatttaaccaatttgcaccatgtgtaatatgtaagagatcaaaaattatattgagttttatttatcttttaactgatatgggaaacatttttatagataatttttattcaagttctaagttacttcatttttatttcggcgaagttggtgcttttttttcttttcaaaccgtttgaagatggtttcctcatggattggagtttaatccatttttctatcatgagctc

SEQ ID NO: 2 - DNA, 3568 bp, Lotus japonicus
  5′UTR (1)..(137)
  CDS (138)..(3119)
  3′UTR (3120)..(3568)
ttcaactttcaaaacaaagtggatgggatcttcatcttatataaccacacatcaatcatttgtgctacttctccaattttctttagagatgaaatgaagagctaagcagacaagacaagtttatttgtttgttgctgatgggtcttgggttcaagatgcagcagagccaccaccctgtggctttgaagttacatgagcaagctgggagccagagaaagttcactttcattcagaacttcagaaactggtttctaccccttctgtttgtatggttcattgttatggctgcatttggtgcctgcatctaccataaaatggatgctgaaactaaagtcagaaggaaagaggtgctgggtagcctctgtgatcaaagggctagaatgctacaagaccaattcagtgtcagtgtcaaccatgtccatgcccttgccatccttgtttcaaccttccattactacagaaatacttcagccattgaccaggaaacctttgcagaatacacggccaggacagcatttgaacggccattaatgagtggggtggcctatgcacagagagtggttcactcagagagagaaagatttgagaagcaacatgggtgggttataaagacaatggaaagagtgccttcaggggttagggatgagtatgcagcagtgatatttgcacaggaaactgtctcttaccttgaatctattgatatgatgtctggggaggaggaccgagagaacattttgagggctagagccactgggaaagctgttctgactagccctttcagactgctggattctcatcatcttggcgtggttctaacatttcctgtttataaatctaagctccctccagagccaacgacggaagaggtcattaaagccatagcaggatatattggaggatcctttgatgttgagtcccttgtggagaatttacttggtcaacttgctggtaaccaagcaattttggtgaaggtatatgatataacaaactctagcgaccccctaatcatgtatggcagccaatatgaagagggtgatatgtctcttgtccatgaaagtaagcttgattttggagatccatacaggaaacatcacatgatctgtagatatcaccaacaggcaccaacaaattggatagcatataccacggcattcctattctttgtgattctttgtttagtgggttacattttatatgctgctggaactcacattgtcaaggtagaagatgattacaatgcaatgcaggatttaaaagtcaaagcagaagcagctgatattgccaagtcacagtttctagctaccgtctctcatgaaattagaactcccatgaatggaattttaggaatgcttggtctgcttttacgcacagaattgagttcaacacaaagagactatgctcagactgctcaagcatgtgggaaggcactaatagcattaataaatgaggtgcttgaccgagctaaaattgaagcaggcaaattagagctagaagcagttccatttgaccttcgttccatacttgacgatgtcctttctcttttttctgagaagtcaagacacaaaggcttagagctggcagtgtttgtttctgacaaagttccggatatagttatgggcgatcctgggcgattcagacaaatagtgacaaatcttgttggaaactctgttaagttcactgagcgaggtcatatatttgttaaagtccatttagctgaaaaaagacagtgcacaatgaatggaaaatgtgagacttttctaaatggaggctgtgatgatgttttgcatgtatctggcagttataatttgaaaacccttagtggatatgaagccgctgatgaacggaacagctgggataattttaagcatcatattgctgacgaagaatttttctttgatgcttcggttaaaaagttggcctctagtgaatcttatgagcaagtcaccttgatggtcagcgtggaggacactggaattgggatttctttctctgcccaagatagtattttcatgccttttgtgcaggctgacagctcaacctctcgaaactatgggggtaccgggatcggcttgagtatcagtaagtgcttggttgaactgatgggcggtcagataaacttcataagccgaccccaggtcgggagcacgttttcattcactgcagatttcggaacatttaagaaaaactcaacaactgacatgaagaaacttaactttgaagatctaccttctagttttagaggtcttaaagccattgtggttgatggaaaacctgttagagctgcagtgactagataccatttgaagagactagggatacaagctaaagttgcaattagcatcaataaggctgtttctttatgtgggaaaaatggttctttgacctcggcattatttcagcctgatattatttttgttgagaaggactcttgggtttctggagaggatggtggtatcttcaatgcgtttaagatgcctcaaatgatccttcttgcaaccaatatctgtaacgctgaatttgataaagccaaagctgcaggtttcagtgatacagtgatcatgaagccactgagagctagtatgctggctgcttgtcttcagcaagttttcgggactggcaagacgaggcagtttgggaaagacatgtcgaatggttcttcagtacgaagccttctttgcggaaagaaaatcttagtggttgatgataatttggtgaaccgaagggtcgccgccggcgcgttgaaaaactttggagctgatgtcaaatgtgcagcaagtggcaaagctgctcttgaaatgcttcaatatcctcacgatttcgatgcttgcttcatggatattcaaatgccagaaatggatgggtttgaggccactcgtcgaattcggatgatggaaagagaggcaagtgagcagctgaaaagtgaatctggtgaagaaaatggtaagaaaagtgagttccacatgcctatattggccatgacagctgatgtaatccatgctacatatgataagtgcttaaattgtgggatggatggatacgtctcaaagccttttgaagaagagaatctctatcaagcagttgcaaagttcttcaagtccaagcctgcttcagactcatgacactgcttattctgcagaacaggtcaaccaacttttgattgagaaacatttagtgttagcatgtttggatcaacttctcccagcatcaattctgaaactcagaagctactcatagaagcttctagccagaattgattttggcttcagaagctactaatggttttatgtagagagcaaaagttggtttccaagatatgcgaggatccatgatgatacacacgtctgaagtgatcatttctaaccagttgaagtttcactcgacgtgatttgaatccaagtaaatgcataccacataattatccatccccatcttgtgtacagattctcccaagggataagaaaatttatgtaaattcaatttttttcttttgcatctcaatacttccctgttagaactttttccctatgattatttccacttttcattttcaattatattttttgtaaaa

SEQ ID NO: 3 - protein, 993 aa, Lotus japonicus
MGLGFKMQQSHHPVALKLHEQAGSQRKFTFIQNFRNWFLPLLFVWFIVMAAFGACIYHKMDAETKVRRKEVLGSLCDQRARMLQDQFSVSVNHVHALAILVSTFHYYRNTSAIDQETFAEYTARTAFERPLMSGVAYAQRVVHSERERFEKQHGWVIKTMERVPSGVRDEYAAVIFAQETVSYLESIDMMSGEEDRENILRARATGKAVLTSPFRLLDSHHLGVVLTFPVYKSKLPPEPTTEEVIKAIAGYIGGSFDVESLVENLLGQLAGNQAILVKVYDITNSSDPLIMYGSQYEEGDMSLVHESKLDFGDPYRKHHMICRYHQQAPTNWIAYTTAFLFFVILCLVGYILYAAGTHIVKVEDDYNAMQDLKVKAEAADIAKSQFLATVSHEIRTPMNGILGMLGLLLRTELSSTQRDYAQTAQACGKALIALINEVLDRAKIEAGKLELEAVPFDLRSILDDVLSLFSEKSRHKGLELAVFVSDKVPDIVMGDPGRFRQIVTNLVGNSVKFTERGHIFVKVHLAEKRQCTMNGKCETFLNGGCDDVLHVSGSYNLKTLSGYEAADERNSWDNFKHHIADEEFFFDASVKKLASSESYEQVTLMVSVEDTGIGISFSAQDSIFMPFVQADSSTSRNYGGTGIGLSISKCLVELMGGQINFISRPQVGSTFSFTADFGTFKKNSTTDMKKLNFEDLPSSFRGLKAIVVDGKPVRAAVTRYHLKRLGIQAKVAISINKAVSLCGKNGSLTSALFQPDIIFVEKDSWVSGEDGGIFNAFKMPQMILLATNICNAEFDKAKAAGFSDTVIMKPLRASMLAACLQQVFGTGKTRQFGKDMSNGSSVRSLLCGKKILVVDDNLVNRRVAAGALKNFGADVKCAASGKAALEMLQYPHDFDACFMDIQMPEMDGFEATRRIRMMEREASEQLKSESGEENGKKSEFHMPILAMTADVIHATYDKCLNCGMDGYVSKPFEEENLYQAVAKFFKSKPASDS

SEQ ID NO: 4 - DNA, 12738 bp, Lotus japonicus
  promoter (1)..(2359): promoter + 5′ UTR
cccgggattgttcatatccccatgtgtgtgtttggtttggacacaagcaagcttgccacagtggtgtttacaactcattctgaattatatatatatatatatatatataactaatctctatcaataatttagttagttttatgggataatatgattcccgtcaaattagcttgtaggttaaaaatccatatttatataataaattagacttatctaaatcacttaagtaaatatgatgtaaaatcacaatagcttaggtagtttaacaatgttttatcatgaatttaaaaaaaattagttgtttcaaaatattattggaccaaataagcaaagaggagctaatccaattataaacagcacatctttataattgttttctactcgatctaatattggttccttctttccttgagaaaaaaagggttaaaaatgcgatttgttttgtaaaattaatttgtgacattaacaagagaaaactctacttttttaatttttaagaagttacttaaatatagaaagaaagaagttacttaaatatgaaacttgtctaaccaaagtcaaccgatcaaagcgtttggttagcaaagagaacttaaaaaaataactaaattaactgtttgaacagtgatgtaacagagaaatagaatggcttacatatggattatggagttaatggaaggcacactagttttagttcacggcaacaagacagaattatcctcctatattttaggaggttttcttatttatttttaatgtaagacgtgtatccttcttttgaggtaataatgttcgagcttgaaacattcacattatagtgtgactaattgcttaatatgaatcaagcatgtaccacttgaaacactcactcatcgtttataatataattactttatagtactataaagtatgtaattattttctcctttttaagtatctcaatcatttttttgacccaaatattattttagagaagaaacatgtcttaagaccaatacatcaacatatatggagagtagtttgacaacctctcatgagctttctaaagccaaacaaatgtactcacataaaaaaattaaaaatttacagcaagtgagttataaagagtgattaataaatgttgaataacgatggagggtagttatgaaattccataaataaagcaaaggatgtttatggcaattgacatgggaataagatccgcgcgttgtcgccgtgtcataatcgctcagatttgtgatagcgagagatttccattcttttcctcttttttcgaacgaacgaacgaacgaacacagtagcagctgtattaggattcagattgcatgatacattgatattgatattgatattgatattgatattgttgtgggtcaagtctctactctactatccaaagtaagcatatatatagagagagagagagagcttgggactagggaggatattagcttatgtgactgtgaagttgaagggagacaagagcgtatctggcaaaatcctcaaaataaaatactagtactagtacagagaaaagagactaagagagagagtgctgctgcacatcaagacccattgtgatttgtgatttgtgatttgtgatttgtgatttgtgagtgagttcattgtacaggtattattgtttgttgtttctctcctcaaccaccctctaaagtctaatctaactcattgggctctgtgcttagctggttgtgttttgtgtatggtgaattagggggtcaatctctggttttcatcattattatattatatatgggaataccgtgctctcttctttgtccttcaccaaaactagtttcacgcctacacaacatgattagagcctcttcatttttttaatctcatcctttaagtgtatttctatttctattggctattgggacaaggggaaggtggtgcttcttaggaacttgagctgttttccatcttttgagacccatgctttgtctctctcatttttaattctgggtctctttcttctcttgtcctgattttttaaatgtgcttcttttttgcttcttacaaccaccctctaaaccattcatcatgcttggtttgcttttgcttctcctttcacaggtttcaatcacgcaaaacaatgctgcaatgatgctgtacttggagtttcttctgtgaccccttttttccttccttcaacaatcaacccaccagagaaaagtgtctcagattttgagactactttcaactttcaaaacaaagtggatgggatcttcatcttatataaccacacatcaatcatttgtgctacttctccaattttctttagagatgaaatgaagagctaagcagacaagacaagtttatttgtttgttgctgatgggtcttgggttcaagatgcagcagagccaccaccctgtggctttgaagttacatgagcaagctgggagccagagaaagttcactttcattcagaacttcagaaactggtttctaccccttctgtttgtatggttcattgttatggctgcatttggtgcctgcatctaccataaaatggatgctgaaactaaagtcagaaggaaagaggtgctgggtagcctctgtgatcaaagggctagaatgctacaagaccaattcagtgtcagtgtcaaccatgtccatgcccttgccatccttgtttcaaccttccattactacagaaatacttcagccattgaccaggtttgtgcttgattttcctttccttgaagcattttttagttggaggctcaatttctttttctgatttgattctggccttaaaaattagaatcaattgtagaaggatttccaaacatgcccattttggaaattggtgcatctgatagtatcatgtttagatcagtttctttttcctcagaattgattttgggcttaaaatcaattgtggaaggatattcattagtaatttggatattgttgcatcatatggttctatctagttacatcatttttttccactctgattgcatgtatctttctcctgttcttttccctatcaggaaacctttgcagaatacacggccaggacagcatttgaacggccattaatgagtggggtggcctatgcacagagagtggttcactcagagagagaaagatttgagaagcaacatgggtgggttataaagacaatggaaagagtgccttcaggggttagggatgagtatgcagcagtgatatttgcacaggaaactgtctcttaccttgaatctattgatatgatgtctggggaggtaaatgtcaacacttgtgaattaattgtaaaactcagaagctactcagagaagctcttccccagaattggttctgcctttagaataaattgtacatggatttgaccacattttctcatttgcatgatgcaggaggaccgagagaacattttgagggctagagccactgggaaagctgttctgactagccctttcagactgctggattctcatcatcttggcgtggttctaacatttcctgtttataaatctaagctccctccagagccaacgacggaagaggtcattaaagccatagcagggtatgtcctcatttcacttttcttgccaaaaccagacttctatttggttgtgtttccgtaggctatgactgatatgtagtttcaactcagttagactataatataaacccttcatgtggcccttacccaacagcttaagcttttgggataattggttgcttgacaaactccttccgtagaaaacttggttagctttggttctatgtgggctttatgttttccctgagcttatgtaatagcatgatgtgtttaatgtactttttaatggaaacagatatattggaggatcctttgatgttgagtcccttgtggagaatttatttggtcaacttgctggtaaccaagcaattttggtgaaggtatatgatataacaaactctagcgaccccctaatcatgtatggcagccaatatgaagagggtgatatgtctcttgtccatgaaagtaagcttgattttggagatccatacaggaaacatcacatgatctgtaggtgggtgcttctagttattgttgtagtacattttctgagtttcagtggtttatcaattatcagcagattcttatgatcaatttttttaacagatatcaccaacaggcaccaacaaattggatagcatataccacggcattcctattctttgtgattctttgtttagtgggttacattttatatgctgctggaactcacattgtcaaggtagaagatgattacaatgcaatgcaggatttaaaagtcaaagcagaagcagctgatattgccaagtcacaggtacttttcatgacatgttagcactgttcgttatttccttgaattgcatactgatcactagaaactgaaaatttgttattaatgtcagtttctagctaccgtctctcatgaaattagaactcccatgaatggaattttaggtaactttaagattctctctcgttctctttccactgaaaagcaacacatgctttcattccatacctgatactttcccattagtgatgctatcgttaaactccttgtcactgtaggaatgcttggtctgcttttacgcacagaattgagttcaacacaaagagactatgctcagactgctcaagcatgtgggaaggcactaatagcattaataaatgaggtgcttgaccgagctaaaattgaagcaggcaaattagagctagaagcagttccatttgaccttcgttccatacttgacgatgtcctttctcttttttctgagaagtcaagacacaaaggcttagaggtacgtttagtcattgctaaatctgttgtgaagtcctgtacaagtggcgtaatttcatagtcctatttccttttcttcaattgatgatcttatttcatattcctctcgtgtttctctctttctatgttgccatgtcgttgggcggtgatgttcctactctgatcccaaattcctgatgtgcaattattccgacttggacttcaatggtttgggaagtatagctggcagtgtttgtttctgacaaagttccggatatagttatgggcgatcctgggcgattcagacaaatagtgacaaatcttgttggaaactctgttaaggttagtggaattttcaaactttatttgcctaatgttgtgtgcaagttgtgtgttggaaatgcgtccttttaacgttataaaatcgtacaagttcgtattctccattgtatacaataacttattagcaaagtacttgttgatatcattactgattaactttaatatcttgcagttcactgagcgaggtcatatatttgttaaagtccatttagctgaaaaaagacagtgcacaatgaatggaaaatgtgagacttttctaaatggaggctgtgatgatgttttgcatgtatctggcagttataatttgaaaacccttagtggatatgaagccgctgatgaacggaacagctgggataattttaagcatcatattgctgacgaagaatttttctttgatgcttcggttaaaaagttggcctctagtgaatcttatgagcaagtcaccttgatggtcagcgtggaggacactggaattgggatttctttctctgcccaagatagtattttcatgccttttgtgcaggctgacagctcaacctctcgaaactatgggggtaccgggatcggcttgagtatcagtaagtgcttggttgaactgatgggcggtcagataaacttcataagccgaccccaggtcgggagcacgttttcattcactgcagatttcggaacatttaagaaaaactcaacaactgacatgaagaaacttaactttgaagatctaccttctagttttagaggtcttaaagccattgtggttgatggaaaacctgttagagctgcagtgactagataccatttgaagagactagggatacaagctaaagttgcaattagcatcaataaggctgtttctttatgtgggaaaaatggttctttgacctcggcgtaagtctttaattaacctttttggtttcaattatgtagaaatgtattgaatgttatgataatcagtagcattatcaacttttagtaattgttcttaacatatgctaatagtcatatctttctataatactacaatactgtagccatataatatctttcctgtattggagtgagttttcaaatgtttttctgtgatattttggaagttatcttcagtttgagaactcatttgtcatttttgcattttgttattggatatttggatggatctttacaaaggatgtgtggattttgacttgttatacacatttcttctccattttatattgtttgtgttattctttttactcataaagaaatttagaaactgcattgactggttctttttaattacttacagatattgacattgatattttttgtaaatgctgtcttgacatggtttaattacttacagactaggtttttctttccttttctaacatgcatatccatttacttttttgaccaaccaacatcctcatgagtcatgacatgttgatgatttatatggttgacttgagactatttagacattaaataaccgcaaattccatgttgtttgtgtgtttggttccctgttgggtaatctcagaatcaattatgatagagtaaaatcaattttggatgagatgtgtgggtgtcattttgtaaacctaaacccaaaatcaattctgctataagctagagagagtagttgaacataatcaattgtgagattttgcaagtggattgcacaacattgccctatgaaaatcactttttgttcacaaaatttatctaaacatacataacttcattttcaacctttactataatcaattttacaataattaattttacccaaaatcaattgtgacaatgagtttccaaacacacacttaaagactaccatttgcagaaaatatgtgatagaagacttatgtttatgtagtgtgtttcagttcattcactgatttaaactactcggattttgcagattatttcagcctgatattatttttgttgagaaggactcttgggtttctggagaggatggtggtatcttcaatgcgtttaagatgcctcaaatgatccttcttgcaaccaatatctgtaacgctgaatttgataaagccaaagctgcaggtttcagtgatacagtgatcatgaagccactgagagctagtatgctggctgcttgtcttcagcaagttttcgggactggcaagacgaggcagtttgggaaagacatgtcgaatggttcttcagtacgaagccttctttgcggaaagaaaatcttagtggttgatgataatttggtgaaccgaagggtcgccgccggcgcgttgaaaaactttggagctgatgtcaaatgtgcagcaagtggcaaagctgctcttgaaatgcttcaatatcctcacgatttcgatgcttgcttcatggatattcaaatgccagaaatggatgggtatgcttactggcactgactaatacatgttttttgccaacttaatatattactctttcaatattcgttgtgttattagaagatcatatagattaatttataaattttcttttagcaaaaccttatcaattaagtgtgtagaaaagtcagtctcacattatggtcaaataagtgttagggcaagcttcacctcaaagctagctatttgggtagatttaggcctaacccgaattctaagatggtatcagagtctatcctagatctttttattggaaaccacccgtatatgagcaactcgtagatattcattcttgaaagttgcacgctccatatgtccattcctaggtgcgagagagaagtctcactttgactagagatatgattaaaaaaatatttataaagggttgagcaatcctcacctcagagctaagcttttggggtaaagttaggcctaactcgaactctaataaagtgtttagctggtgtgtcaactgtcaatatgaaatcttttgcaatttactatgcattcacttacctactttattgaagcttattgacaatttgtgcagaagcatcattaattaggaacatgttagctatacaagttatgatgtttttgtatagcatatcatgttccaaccttccaataacaaaatatgtggttcaagtgtgagaatatataggttaaacaataaagtattgagttaacagaaatctaaacacacgctgtcactagctcttcatattgagacatgcatgggatttgacaaaacatctgaataaatatttgcaggtttgaggccactcgtcgaattcggatgatggaaagagaggcaagtgagcagctgaaaagtgaatctggtgaagaaaatggtaagaaaagtgagttccacatgcctatattggccatgacagctgatgtaatccatgctacatatgataagtgcttaaattgtgggatggatggatacgtctcaaagccttttgaagaagagaatctctatcaagcagttgcaaagttcttcaagtccaagcctgcttcagactcatgacactgcttattctgcagaacaggtcaaccaacttttgattgagaaacatttagtgttagcatgtttggatcaacttctcccagcatcaattctgaaactcagaagctactcatagaagcttctagccagaattgattttggcttcagaagctactaatggttttatgtagagagcaaaagttggtttccaagatatgcgaggatccatgatgatacacacgtctgaagtgatcatttctaaccagttgaagtttcactcgacgtgatttgaatccaagtaaatgcataccacataattatccatccccatcttgtgtacagattctcccaagggataagaaaatttatgtaaattcaatttttttcttttgcatctcaatacttccctgttagaactttttccctatgattatttccacttttcattttcaattatattttttgtaaaattggtctccatcctataaggtttgcttagttttttttaattcagatgagaaggttggtgcttataatgtgtatacctttttagcagtacttgtgatataaatatttgtcttatttttagaacttaggagataataagatcgtaggagtaaatagcatttttggagaatgatatcataattataatcatctacattctgcagttataaaaaaaatcaatattaatgcatagcaagtagctttaagaagtatgatctatttaagtattgaattttttttctaaactaattacaatgtattattgctgggatatgcgctttttactcgatagatatcatgtttggattagtttttcttagaatccattttggaaccgaaaaaattatcacataatttattcttcaaagttgattttggcaatcaaggtaaacactcataaaagctaaagggagttgtttcttattattgtgattttagctcattgtggtttttcaccgtgtatataaacatgcatttgatgtgacgttgacttgtgaagcacgtggctatccaccagcaatctcaagtaacacttgtcagcactatgttgctattataagtgacaaggcagtgaccacacccctctatatatgcataaacttttttattaaaagtaacagaatcaaaagggaagttttctacaaattaattaatggaaaattatcagaaccagtatggagctgttcctgctagcagggctccggttcagagccagagtgaaatattggaaaccaccaatgttggcggtgctacacaagtcataagaaatgattatgatggaagaaccactgctgcagatgctactgttgttggtgagagcgatctgcagcatcaccacaagaaaggaatattagagaagatcaaggagaagcttcctggaacacaccaccatcaagatcacaaatagctagctagctttgtttagtttattttgcttctatcattataaatgtaataataatagtatgtgtgttgctacatgcatgtgtatgtatcgtttagtcagctagtttgttaataacttgctttcaattttgcttccttacaagcctatttatgcaatgtatttggtcatttcactattggtttctaatcaatctaaattagtacatggtcctcttccttttagtgccttttaattaaagagtaaatgttgaatattgtttttagtcattctatttacatttttattttttttgcttcatcggaaaattatttacatttttattaagtgtgtgtttagattttcgtttttcgttaaaaatttcataattaattttttataacagtgatgtatgcagctaaggcatcctaacagacctatctttgtatgtatggcattctgggcgggtcggatttatttttagccccataattccccactactatacataaaatgatacctgctagtaaaataatttaaagtgtttcgaagtcatattggtaaattgatttcagttaaaaatcgaagtttgttagctactaaagacggtagcttctatgtcaaaatagttaattcctatattttataattggatcattttttctaaaatttattcaaacatctcaactcactttgactataatcactttataattaattttgttgaattaaataaattatattcaaattaaatgtgataatacccactgaatgtgattggaatgccatcagggaaatatggaacaaagagagctgcatgtggaagggcttgttatgtctgcaaaacagacaaagaagttgtaatcacaattaggacaattgccaaagatgtggggtcacattgacttgtcagggaaagtctatagatccactgtgctcttaaagctagaacaattaacataaaatttctgccaattaaggctttcacttcctatccctatctgctgtatattttaagtttgagtaaagggacagttgtggtgatgttataatcaactgcaggctgagggaaaagttgggggtcaagtggtgtctatataaacttttttgtcattattaatctttaatctttttggttttgtaaaatgacagcttttgtcttgtgctgaaagttgactgtgctagctagtaaagtcatgctggtttcttaattgattgtggtgtttttatgcgacaactgtagacattgcaatgcaggcacacaagtcaagaggcaattacaaacaaaatacagctattaataattgacctactaactactaaggagggaatttaaatgtgattttgaaatcttatattggatgtaaagaggcaatactcatcagactgtaaagtgacagaacaagtagatggtaaacaagtagaagtagaagcagcaacaaattgagtaaagaccatcaatgtcaacacttccttggagaagaatttcataagtgtcatgtgaaaataactagacaaaatgtagccaaaatgaagtatgaaactcagttattgaaacgagtgacactgactagattccaagttattgaaggaaccagcaaaaggttaagtgacagtgaagtgtgtgggttgaaagaggattgaataaaagctgaaccaatggactgaataggcaaacttgacaatttcccattagaatctgttttaacttccttgtacaaaagtttcttcggtgaaatttccagcattgttggtaacatgatgagtagctcatgaatctggaaaccaaacagttttgtttgttcaagctgtagagaagaatgagaactacatgtgagcattgttatgtaccaaaaaaacctgtgagcattgttgctgcaatcctggatcagtacgcatagcctgagcacaaagatgttgaattgaagtagttgctctaatatgttatgaatattcaagattaaaacatttttaagttatcatgtgacaatcacataactattaaattattttaatcttaactattaatcattagatctaacgatcatattctcatataaaataattattctgatacaataatatatatatatatatatatgtatgtaggggatctgaggatgtatcatatgagaaatgtgatattatgtgagaacatgagaataaatcacaaccgttagatttaatcaacggaccagattaaaacaatttaatgctcacatgaccacttaaaaatgtcatgtgactttcttaaaaagtcaaatgacttcatgttttaattttgaccgttcatgattagatttaacggtcatgatttattctcatgttctcacataaaaacatttttcatttttctgatatgtatatttgtaacataaaacacaacataaaatactatgtaaatcagccaatttcaccaaatacaatatgatgtgaaaaatataatattactattgatattcactcaaatgcacaatcggccctaaaaaatggtcaatttttctggttaatatatttcttttttaaaacatgagtattcatgcaatatatgatttcaaaatatataatttctttcaacatttaaccaatttgcaccatgtgtaatatgtaagagatcaaaaattatattgagttttatttatcttttaactgatatgggaaacatttttatagataatttttattcaagttctaagttacttcatttttatttcggcgaagttggtgcttttttttcttttcaaaccgtttgaagatggtttcctcatggattggagtttaatccatttttctatcatgagctc

SEQ ID NO: 5 - DNA, 3568 bp, Lotus japonicus
  5′UTR (1)..(137)
  CDS (138)..(3119)
  3′UTR (3120)..(3568)
ttcaactttcaaaacaaagtggatgggatcttcatcttatataaccacacatcaatcatttgtgctacttctccaattttctttagagatgaaatgaagagctaagcagacaagacaagtttatttgtttgttgctgatgggtcttgggttcaagatgcagcagagccaccaccctgtggctttgaagttacatgagcaagctgggagccagagaaagttcactttcattcagaacttcagaaactggtttctaccccttctgtttgtatggttcattgttatggctgcatttggtgcctgcatctaccataaaatggatgctgaaactaaagtcagaaggaaagaggtgctgggtagcctctgtgatcaaagggctagaatgctacaagaccaattcagtgtcagtgtcaaccatgtccatgcccttgccatccttgtttcaaccttccattactacagaaatacttcagccattgaccaggaaacctttgcagaatacacggccaggacagcatttgaacggccattaatgagtggggtggcctatgcacagagagtggttcactcagagagagaaagatttgagaagcaacatgggtgggttataaagacaatggaaagagtgccttcaggggttagggatgagtatgcagcagtgatatttgcacaggaaactgtctcttaccttgaatctattgatatgatgtctggggaggaggaccgagagaacattttgagggctagagccactgggaaagctgttctgactagccctttcagactgctggattctcatcatcttggcgtggttctaacatttcctgtttataaatctaagctccctccagagccaacgacggaagaggtcattaaagccatagcaggatatattggaggatcctttgatgttgagtcccttgtggagaatttatttggtcaacttgctggtaaccaagcaattttggtgaaggtatatgatataacaaactctagcgaccccctaatcatgtatggcagccaatatgaagagggtgatatgtctcttgtccatgaaagtaagcttgattttggagatccatacaggaaacatcacatgatctgtagatatcaccaacaggcaccaacaaattggatagcatataccacggcattcctattctttgtgattctttgtttagtgggttacattttatatgctgctggaactcacattgtcaaggtagaagatgattacaatgcaatgcaggatttaaaagtcaaagcagaagcagctgatattgccaagtcacagtttctagctaccgtctctcatgaaattagaactcccatgaatggaattttaggaatgcttggtctgcttttacgcacagaattgagttcaacacaaagagactatgctcagactgctcaagcatgtgggaaggcactaatagcattaataaatgaggtgcttgaccgagctaaaattgaagcaggcaaattagagctagaagcagttccatttgaccttcgttccatacttgacgatgtcctttctcttttttctgagaagtcaagacacaaaggcttagagctggcagtgtttgtttctgacaaagttccggatatagttatgggcgatcctgggcgattcagacaaatagtgacaaatcttgttggaaactctgttaagttcactgagcgaggtcatatatttgttaaagtccatttagctgaaaaaagacagtgcacaatgaatggaaaatgtgagacttttctaaatggaggctgtgatgatgttttgcatgtatctggcagttataatttgaaaacccttagtggatatgaagccgctgatgaacggaacagctgggataattttaagcatcatattgctgacgaagaatttttctttgatgcttcggttaaaaagttggcctctagtgaatcttatgagcaagtcaccttgatggtcagcgtggaggacactggaattgggatttctttctctgcccaagatagtattttcatgccttttgtgcaggctgacagctcaacctctcgaaactatgggggtaccgggatcggcttgagtatcagtaagtgcttggttgaactgatgggcggtcagataaacttcataagccgaccccaggtcgggagcacgttttcattcactgcagatttcggaacatttaagaaaaactcaacaactgacatgaagaaacttaactttgaagatctaccttctagttttagaggtcttaaagccattgtggttgatggaaaacctgttagagctgcagtgactagataccatttgaagagactagggatacaagctaaagttgcaattagcatcaataaggctgtttctttatgtgggaaaaatggttctttgacctcggcattatttcagcctgatattatttttgttgagaaggactcttgggtttctggagaggatggtggtatcttcaatgcgtttaagatgcctcaaatgatccttcttgcaaccaatatctgtaacgctgaatttgataaagccaaagctgcaggtttcagtgatacagtgatcatgaagccactgagagctagtatgctggctgcttgtcttcagcaagttttcgggactggcaagacgaggcagtttgggaaagacatgtcgaatggttcttcagtacgaagccttctttgcggaaagaaaatcttagtggttgatgataatttggtgaaccgaagggtcgccgccggcgcgttgaaaaactttggagctgatgtcaaatgtgcagcaagtggcaaagctgctcttgaaatgcttcaatatcctcacgatttcgatgcttgcttcatggatattcaaatgccagaaatggatgggtttgaggccactcgtcgaattcggatgatggaaagagaggcaagtgagcagctgaaaagtgaatctggtgaagaaaatggtaagaaaagtgagttccacatgcctatattggccatgacagctgatgtaatccatgctacatatgataagtgcttaaattgtgggatggatggatacgtctcaaagccttttgaagaagagaatctctatcaagcagttgcaaagttcttcaagtccaagcctgcttcagactcatgacactgcttattctgcagaacaggtcaaccaacttttgattgagaaacatttagtgttagcatgtttggatcaacttctcccagcatcaattctgaaactcagaagctactcatagaagcttctagccagaattgattttggcttcagaagctactaatggttttatgtagagagcaaaagttggtttccaagatatgcgaggatccatgatgatacacacgtctgaagtgatcatttctaaccagttgaagtttcactcgacgtgatttgaatccaagtaaatgcataccacataattatccatccccatcttgtgtacagattctcccaagggataagaaaatttatgtaaattcaatttttttcttttgcatctcaatacttccctgttagaactttttccctatgattatttccacttttcattttcaattatattttttgtaaaa

SEQ ID NO: 6 - protein, 993 aa, Lotus japonicus
MGLGFKMQQSHHPVALKLHEQAGSQRKFTFIQNFRNWFLPLLFVWFIVMAAFGACIYHKMDAETKVRRKEVLGSLCDQRARMLQDQFSVSVNHVHALAILVSTFHYYRNTSAIDQETFAEYTARTAFERPLMSGVAYAQRVVHSERERFEKQHGWVIKTMERVPSGVRDEYAAVIFAQETVSYLESIDMMSGEEDRENILRARATGKAVLTSPFRLLDSHHLGVVLTFPVYKSKLPPEPTTEEVIKAIAGYIGGSFDVESLVENLFGQLAGNQAILVKVYDITNSSDPLIMYGSQYEEGDMSLVHESKLDFGDPYRKHHMICRYHQQAPTNWIAYTTAFLFFVILCLVGYILYAAGTHIVKVEDDYNAMQDLKVKAEAADIAKSQFLATVSHEIRTPMNGILGMLGLLLRTELSSTQRDYAQTAQACGKALIALINEVLDRAKIEAGKLELEAVPFDLRSILDDVLSLFSEKSRHKGLELAVFVSDKVPDIVMGDPGRFRQIVTNLVGNSVKFTERGHIFVKVHLAEKRQCTMNGKCETFLNGGCDDVLHVSGSYNLKTLSGYEAADERNSWDNFKHHIADEEFFFDASVKKLASSESYEQVTLMVSVEDTGIGISFSAQDSIFMPFVQADSSTSRNYGGTGIGLSISKCLVELMGGQINFISRPQVGSTFSFTADFGTFKKNSTTDMKKLNFEDLPSSFRGLKAIVVDGKPVRAAVTRYHLKRLGIQAKVAISINKAVSLCGKNGSLTSALFQPDIIFVEKDSWVSGEDGGIFNAFKMPQMILLATNICNAEFDKAKAAGFSDTVIMKPLRASMLAACLQQVFGTGKTRQFGKDMSNGSSVRSLLCGKKILVVDDNLVNRRVAAGALKNFGADVKCAASGKAALEMLQYPHDFDACFMDIQMPEMDGFEATRRIRMMEREASEQLKSESGEENGKKSEFHMPILAMTADVIHATYDKCLNCGMDGYVSKPFEEENLYQAVAKFFKSKPASDS

SEQ ID NO: 7 - DNA, 4743 bp, Medicago truncatula
  exon (1)..(348)
  Intron (349)..(462)
  exon (463)..(696)
  Intron (697)..(799)
  exon (800)..(969)
  Intron (970)..(1261)
  exon (1262)..(1480): mutated codon encoding L/F substitution
atgggtcttctcttgaagatgaagatgcagaatcagcaccaccctttggcttctaagttacaagaacaaacggggaacaaaagatacacattcattcaagcacatagagcttggcttctcaaattaatgtttctatggattcttctgatggctctgattagtcgtatcatctacagcaaaatggatgtgggtactaaagtgagaaggaaagaggttttgggtagtctttgtgatcaaagggctagaatgttgcaagaccaattcagtgttagtgtcaaccatgttcatgctcttgccatccttgtttcaactttccattattacagaaacccttctgccattgaccaggtttgtgcttgaaagttttgatcattctgtgttggaaatgaaaaatactcaatctttgttgtgtttttgaacctttatgtatcctctgatgattatttgatgatttcccttcaggaaacttttgcagaatatacggctaggaccgctttcgaaaggccgctacttagtggagtggcctatgcacaaagagttgttaactcggaaagagagcagtttgagaagcagcatggagtggttataaagacaatggaaagagaggcttcaccggttagggatgagtatgcaccggtcatatttgctcaggaaactgtctcttaccttgagtctattgatatgatgtctggagaggtaaagaacgacacttgtgaaccattccaccgcttgtttttttttttttttttttggtggtggattacttttattactacaatttctcatttttgcaatgcaggaggatcgagagaacataatgagagctagagctactgggaaagctgttctgactagcccttttaggttgttgggttctcatcatctcggtgtggttttaacatttcctgtttacaaatctaagctccctcccaacccaacaacagaagagctcattaaagcgaccgcagggtatatgcttatttcactaattgtcagctttttgtttttagaatcttttttcttatgtttcatattagtaattagaggatgaaggttgtgtttagtataaaaattttaggtacattattagcattttattaagcagcagttaatctgtcaatgccactattagtgttatcgacacaacttctatcattgagttgaagaaaactgtttctgttacaaggcttgtatttggttatattttgttgtaagcatttcaatagaacatgtgattaatgaactttttaattggaggcagatatgttggaggatcctttgatgtggagtcacttgtggaaaatttatttggtcaacttgctggtcatcaagcaattttggtcaatgtatatgatgtcacgaactcttctgatcccctaatcatgtatggcaaccaatacgaagaaggtgatgtttctcttgtccatgaaagtaagcttgattttggagatccatacaggaaacatcaaatgatatgtaggtaggttcttctggctactgtacattacattttctggaactgactttcggcattttgtcagcagaaagatgcttatgatcaaacttttgtacaggtatcaccagaaggcaccgccaaattggacagcactttctactgcaatcctattctttgtgattcttcttttaatcggttacattttatatggtgctgggaatcatattgtcaaagtagaggatgatttccacgaaatgcaggagctaaaagttcgagcagaggcagccgatgttgccaagtcacaggtacttttccttgacatgtcgttggcacttggcatatttcgatatttcctttgattacgcaacaataactaatactgaaatttttgtttttgtcagtttctagctactgtctctcatgaaattagaacacctatgaatggcattttaggtaactgcaaaattctctctctctttgcctaacatgtaacatatgctttcatagctttcacttgctcattaataatgttatcacaaaacaaaactctgtatgattgtaggaatgcttggtctgcttctacgcacggaattgaactcaactcaacgggactatgctcagactgctcaagcatgtgggaaggctctgatagcattaataaatgaagtgcttgaccgagcaaaaattgaagctggcaaattagagctggaagcggttccatttgaccttcgttccatactcgatgatgtcctctctcttttttcagagaagtctagacacaaaggtttagaggtatgctcaatcgttactaaacctattgtgaaggccaatacgagtttcataataatatcttaattcggtttcctctctttatttgatgatctaacttcattatcctttttttttttttcattttacctttcatagctggcagtgtttgtttctgataaagttccagatattgttatgggagatcctgggagattcagacaaattgtaacaaatcttgttggcaactctgttaaagtaagtggaattttcaaattttatttgcctaaagttatttgcattactaatattcatcttatgttggaaatgtttgaaattttgctgttgtatagcaagatacttgttgattacatttctgattaacttcaatatattgcagttcactgagcgaggccatatatttgttaaagttcatttatctgaaaacagaaagcccgtaacaaatggaaagcatgagacttatcgaaatggagggtctgaagaagttgtgcatgcatctggcggttataatctcaaaacgctaagtggatatgaagctgctgatgaacgcaacaactgggataattttaaccacttaattgctgatgaagagtttttctgcgatgcttcaactaaaaaagtggcctcgaatgaattttatgaacaagtcaccttgatggtctgtgtcgaagacactggaattggaattcctttctcggcccaagataggattttcatgccttttgttcaggcagatagctcgacttctagaaattatggtggtaccggcattggtttgagtatcagtaagtgcctagttgaactaatgggtggtcaaataaactttataagccggccgcaggttggaagcaccttttcatttactgcggattttggaatatttaagaagaatccaataactgaggtgaagaaggttaactatgaagatctaccatccagttttagagggcttaaagccgttgtggttgatgggaaacctgttagagctgctgtgactagataccatttgaagagacttgggatacaagttaaagtcgcaaatgccatcaataaggctgtttccttgtgtgggaaaaatggggcttccagcacagggtaagtttttaattttcctttttgtaattaatgcattgctatttttaatgaattaatgttaggactatgtgtgtttaatcataattccatctaaagtcacacatgataggctgtgacttgctgacagtgtaaaacgtataaaacattatacactaacagtgtatcaaaattaaactctttgaagaattgcatttgttcttgttgtgtggttcaatacttgagttcattaactgagttaaaatatttggttgtggcaggttattccagcctgatattatttttgttgagaaagattcatgggtttgtggagaggacgggatcttcagtgtgcgccaactggactggaaacagaatggacatatatttaagatgcctcaaatgatccttctagctacaaatattagtaatgacgaatttgataaagctaaatccgcaggttttagtgatacggtgatcatgaagccactgagagctagtatggtgggagcttgccttcagcaagttttgggaacaggcaagaagagacagctgggaaaagagatgcctaatggttcaacttcagttcgaagccttctgttcggaaagaaaattttagtggttgatgacaatgtagtaaaccggagggtggctgcaggtgccttgaaaaactttggagcggatgtgaagtgtgcagatagcggcaaagctgctcttgaaatgcttcaattccctcacaagtttgatgcttgcttcatggatattcaaatgccagaaatggacgggtatgtttgtttgcatcgattattaattttttttttggtattatttgaacataacatgttaacatgatagttgcattgctgataataaaaaaaaaaacttgcataagttgaatgcaagttacgtgatagttgccctgctgataaattcaatgatatgcacgtaagttgaatgcacgtaacttgcatgttggctatactaattcacattcaaattattttgtttgtatataaactctaattacacatgttatgtatgttctaattcacatccatactatatttgtttgtatataaaccttatcaatagatgttatgtgtgttctaatccacatgaatattatttttacgcatattgttttttataaatcatgtacttgcattattttcaggtttgaagcaactcgtcgaattcgggagatggagaggacagcgaatgaggagacgaatagtgaatgtggtgaaaggaaaagtgaattccatttacctatattggccatgacagcagatgtaatccatgctacatatgaagagtgtttgaaatgtgggatggatggttatgtttcaaaaccttttgaggaagagaatctttatcaagcagttgcaaagtttttccagacaaaacctacttcagtagattcatga

SEQ ID NO: 8 - DNA, 3694 bp, Arabidopsis thaliana
  5′UTR (1)..(68)
  CDS (69)..(3311)
  3′UTR (3312)..(3694)
  misc_feature (3594)..(3594): n is a, c, g, or t
aaaaaatctcactaaaacaaaagaagaagaaagaagaaagaaaatggaatacctacatttttgaagtgatgagaagagattttgtgtataataataatgcaatgttcaatcctctcacaactcattacagctcagatatgaactgggcactcaacaatcatcaagaagaagaagaagagccacgaagaattgaaatttctgattccgagtcactagaaaacttgaaaagcagcgatttttatcaactgggtggtggtggtgctctgaattcgtcagaaaagccgagaaagatcgatttttggcgttcggggttgatgggttttgcgaagatgcagcagcagcaacagcttcagcattcagtggcggtgaagatgaacaataataataataacgatctaatgggtaataaaaaagggtcaactttcatacaagaacatcgagcattgttaccaaaagctttgattctgtggatcatcattgttgggtttataagcagtgggatttatcagtggatggatgatgctaataagattagaagggaagaggttttggtcagcatgtgtgatcaaagagctagaatgttgcaggatcaatttagtgttagtgttaatcatgttcatgctttggctattctcgtctccacttttcattaccacaagaacccttctgcaattgatcaggagacatttgcggagtacacggcaagaacagcatttgagagaccgttgctaagtggagtggcttatgctgaaaaagttgtgaattttgagagggagatgtttgagcggcagcacaattgggttataaagacaatggatagaggagagccttcaccggttagggatgagtatgctcctgttatattctctcaagatagtgtctcttaccttgagtcactcgatatgatgtcaggcgaggaggatcgtgagaatattttgcgagctagagaaaccggaaaagctgtcttgactagcccttttaggttgttggaaactcaccatctcggagttgtgttgacattccctgtctacaagtcttctcttcctgaaaatccgactgtcgaagagcgtattgcagccactgcagggtaccttggtggtgcgtttgatgtggagtctctagtcgagaatttatttggtcagcttgctggtaaccaagcaatagttgtgcatgtgtatgatatcaccaatgcatcagatccacttgtcatgtatggtaatcaagatgaagaagccgacagatctctctctcatgagagcaagctcgattttggagaccccttcaggaaacataagatgatatgcaggtaccaccaaaaggcaccaataccattgaatgtgctcacaactgtgccattgttctttgcgattggtttcttggtgggttatatactgtatggtgcagctatgcacatagtaaaagtcgaagatgatttccatgaaatgcaagagcttaaagtgcgagcagaagctgctgatgtcgctaaatcgcagtttcttgctaccgtgtctcacgagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaggcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtgtctgaagaaatgatcgttgtttccaaacagtcaagttacaacacattgagcggttacgaagctgctgatggtcggaatagctgggattcattcaagcatttggtctctgaggagcagtcattatcggagtttgatatttctagcaatgttaggcttatggtttcaatcgaagacacgggtattggaatccctttagttgcacaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacggatttgaagcaactcgtcagataagaatgatggagaaggaagctaaagagaagacgaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcgtcgtaatccaatcttccggcgagtttttttctctccgcagccggaagaatggactgcttctgctgattgattgggataaatatgcattttggtttctgtacatatagtaggttcacaatctagagattttgaaggttttttttaatttctcacttaagtaatgtagcttgccatgactagtgtatgttgttaaacgacgacgtctaagatggttcagtgttgatcttagcgtaagtattaatcccatgggaatcggttgtactgtatcagatttggttagtcgtttaancattgtaatgttctaataatcactttttcatatatancctcatgttataacatgagacgagaccattttgattaaaaaaaaaaaaaaaaaaaaaaaaaaa

SEQ ID NO: 9 - protein, 1080 aa, Arabidopsis thaliana
MRRDFVYNNNAMFNPLTTHYSSDMNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLFGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKGIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKEAKEKTNLEWHLPILAMTADVIHATYEECLKSGMDGYVSKPFEEENLYKSVAKSFKPNPISPSS

SEQ ID NO: 10 - DNA, 5474 bp, Oryza (rice)
  exon (1)..(348)
  Intron (349)..(711)
  exon (712)..(945)
  Intron (946)..(1040)
  exon (1041)..(1207)
  Intron (1208)..(1508)
  exon (1509)..(1727)
  Intron (1728)..(1818)
  exon (1819)..(2005)
  Intron (2006)..(2087)
  exon (2088)..(2139)
  Intron (2140)..(2233)
  exon (2234)..(2463)
  Intron (2464)..(2694)
  exon (2695)..(2793)
  Intron (2794)..(2910)
  exon (2911)..(3641)
  Intron (3642)..(4079)
  exon (4080)..(4595)
  Intron (4596)..(5215)
  exon (5216)..(5474)
atgggtgtgggaggaggcggaggaggaggaggaggggaggcggcggcggcggtggcggtggagggggatgaggcggggaaggggaggaggtggtggagggtgaaggtgaagctgagcacggtggcggtggtggcgtgggtgctggcgtcggcggcgctctgggcggggctgcactggcgcttccgccgcgcggcgctgcacaaggccgaggaggccctcgtctgcatgtgcgaggagcgcgcccgcatgctgcaggaccagttcgccgtctccgtcaaccacgtccacgccctcgccatcctcgtcgccaccttccactacgacaagcaccctcccgccctcgaccaggtcggcccgaactccgacgagctcttccgccgccgccgcgatgatcctgttgcatctgttgtttttttgccccccgcggttaattgcgataatgcctcgatttttactccacatcttgcccgtgtacttcgctctgctgcttcttcggcttcatttaattctaccgtgaccttccgtgtcagccatggaagccatggattactgttgctgtctcttgctattatatggagcgcactttttgttgggagggagtgaattgattgtgcttgcttgcttctgttggtagtagtactagtgatttctttggctgtgtggctgatgaatcttcttcgatgtgttgtgcgtgcgtgcgcgtgtgtgcaggacacgttcgccgtgtacgccgcgaggacgtccttcgagcggccgctgctgagcggcgtggcgtacgcgcagcgggtggtgcacgccgacagggagagcttcgagcggcagcaggggtggatcatcaagaccatgaagcacgagccgtccccggcgcaggacgagtacgccccggtgatctactcccaggagaccatctcctacatcgagggcctcgacgtcatgtccggcgaggtgcgtttcttgggttacagcttcgcagctgctgctgcggttatcgccatgtccgctgctctgaactgtgctgctgggtgtgcttcggcctgcaggaggacagggagaacatcttgagggcgagggcgacagggaaggccgtcctcacgaggccgttccggctgatgtcgaatcacttgggtgttgtcttgacgtttcctgtctacctcgtcgatctcccaaatgataccgcggtggaggatcgtgttgctgctactgcagggtgagggattactttacttttctgaatgaagattattctctccaactgattcctcttctgtctggaatccactgccttcagctcttcgttttgttgcagtcgttgttggatgcttttagtagtggaaatgtgtgcgtttcagggatatttgatcacatgcaacattttcactcataactggctgaaaaagttttgcattaatagagctgaaatgtctagatggataagcaattgcagtggtattttaagtacaacatgtgcaacgaatggctccatttaactttttctttttgttcggcagataccttggaggagcatttgatgtggagtcactagtagaaaatttgtttagacagttagctggtaaccaggaattggtggtcaatgtttatgatgtcacaaaccactcaaaccctcttgtgatgtatggatctgaggttcctcttggtattccctcaccatcacacacctatacgttggattttggtgatccattgagaaagcatcagatggtttgcaggtaaatttgtgtgaattgatcgttggttttcccattttatattatagaacgatcggtttttttaacatccattggccataaatctgagcagatacagaaacaagcttcatgtttcatggtctgcaattactacaccatcaggggtctttgttatatgtatgctggtgggctacataatatatgctgcttggagtcgctacgataatgttaaggaagattgccggaaaatggaagcgctgaaaaaacgggcagaagcggctgatattgctaaatctcaggtatagttggatgttgtttgcttctctatttctattgcaagcttattgttatatctaaaaggttcttattcatttatgacagttccttgcaactgtttctcatgagatcagaacacccatgaatggcgtgctgggtattttctttgatcttacaacacattcagtttaatgttatgcaactcatttcttttgaaaaaatggaatcatctctttgtttcttttccctaggaatgcttgatatgttattagacacagagctgaagtcaacccagagggattatgcacaaacagcccaagtctgtggaaaggcattaatatccctgattaacgaagtgcttgacagggccaaaatcgaggctggcaagatagatctcgagtcagtaccatttgacctgaggtccatccttgatgatgtcatctcgttattttcttcaaaatcaagagagaaaggaattgaggttagttaaactgatttcggtcatggttggacaaagatcactaaacgtattaagtttctgccagccatcaattatttcttttaggaaaatatcatgcactagttccaccgacatcttttagtctcttagcttgatactctttccatgaacttctctgcattaccgtcatgcaccatgcacgtttaactttgtttaatcccagttgattttcttctatgttgtaacttccagcttgctgtatatgtttctgaaagagttcctgaaatcctgttgggcgaccctggaaggtttcgtcagataattacaaacttggtgggaaactcgatcaaggtaaatgcgcataacctttgtatccattcatgattttctttaacgataccaatagttctcaccaatgacatcaggcaacttgtttcttagtatactattgttcaatgtgaacacaagataacaatatttaccttgtcgcagttcacagaacgggggcacatttttgtacaagttcacctggcagatcactcaaatcttgcaacagaagcaaaaattgaaccagtagtcaatgggatgaatggacataaagacgaggctattgctatacccaccagtgggtctcataacactttaagtggttttgaagcagctgatagccgaaataactgggaaaacttcaagcttttgctctcttacgagaaaaatgaaatgccatatgaaagtgattctgataaagtaactcttgttgttagtgtggaagatactgggataggcataccactgcatgcccaaggccgggtcttcacgcctttcatgcaagctgacagctcaacttctaggaactatggtggaactggcattggattgagcatcagcaaatgtcttgttgaaataatgggtggtcagataaactttgtcagccgacctcttgttgggagcacattcacattcactgctgttctgagaaggtgtgacaaaaatgctattagtgacagtaagactgttgctttgcacccattaccgtccagttttaaaggcttatctgcgctattggttgataaaagacctgtaagagcaactgtgactaagtatcatttgcaaaggcttggaatcacttctgaagttgttggtaccattgatccgacatttggtgtgttgtctgggagaaatggcagttctctaaccaggtacttctatcttctacattcctttcaaaaaattgaaatcctggagttaataggctacttttctctggaaattagaataaacggagcatgcttgcatactaacttcttatgcaaatatcactgttctatgtataaatgattacacgataatgattgttttttggtaagtattactgggtaatacttggccatcatagttcttgcctttattttgtatcacttcggtatattgctacttctgcggcagttctctttacgctaccgaatgtgatatatttaattgaaaattgattttattttaatctgtgaaaagaacatttttttaaggccccacaattctcaaataactagtaagctgattgcagatgatatttgaactatcttgacacccagtttttttttttaatctaactcgttttgttccaaatttgttgtcagcattggtaagaagcagccatgcatgttgctaatcgagagtgattcctggggaccacagatggatgtctccttacatgctagacttcaggagatgaaacagagtgatcgcatacatgtattgcccaaggttttccttctttctgctgcagaatcagacaaagtaaagaagatacatgcagttgattctgtgataccaaagcctctgaaagcaagtgcacttgcggcctgtctgttccaagcacttggtatcacacagccgagccatgagaaacgtgacgattcaggttctcttcatgggcgtgatggttcaggttctcttcatgggttgcttcttggcaagaacatattggtagttgatgacaacaaggtaaacctcagagtggccgctggtacattgaagaaatatggggcaaaggtggagtgtgtggagagtggaaaagatgctctttcccttctacaagtgccgcacaagtttgatctgtgtctcatggacattcagatgccggagatggatgggtaagcttatgtcccgttcaagatttatttcttttgaatttgagccttttatttcatatgatccaaagcgtgacatgtcatctggataagtgccacctcgttgactatcaatttattcacgcaatgaatcaggctttctgcctctttttgaagaaaaaaaaaatgttattcacacttcgtggttaatggaaacaagcatatacattgctgacgccaaatactgattataactagtaaaaatgtcagtcttgtgttagttttttatgtgcgaaaaatgccgtgtctaattcattatgtgaaaattttcaaagacattagccctgatatcgtcctgcattatgccaaacagtactttaaatattaatatcagaaggtctaaaatccgtgcagttaccatttgttcattgtagttttaaccatcatactgcaatatgcagttcttgggctaatgaacataatggtgtgctaacaccctcatgaccaattaacttatgtctcttgaacacttgctgcatattgacatctctgttcctatattttctgaatagtaaccaagtaatgtcaaaccatgtcattattttgtcttcgtttaatcaacatagttttgcttcatgtgctagatttgaggcaactcggcaaatacgagcaatggaagggaaggcaaatgagcaggcagacgacagcgaatcgggttcagaaatcgcagcaaagacggccaaatggcacttgccaatcctggcaatgaccgctgatgtcatccaggccacccacgaggaatgcacaaagtgcgggatggatggctacgtctcgaagccctttgaggagaagcagctcttccaggcagtacagaagttcttgggcccatgcgtttccagctga

SEQ ID NO: 11 - DNA, 3046 bp, Zea mays
  5′UTR (1)..(15)
  CDS (16)..(3003)
  3′UTR (3004)..(3046)
ggggtggagcctgggatgggggtgggaggcggaggcggaggggaggccgctgcggtgtcggcgccggcgccggcggaggaggcggggaaggacgcggaggatggcggcggctggaccttgaaggcgaagctgatcgccgtagcggtgctggtgtgggtgctgggggccttggcgctcggggtgttcctgcactcctacttccgccacgcggcgctgcgcaaggcggaggaagggctcgtcagcatgtgcgaggagcgcgcgcgcatgctgcaggaccagttcgccgtctccgtcaaccacgtccacgccctcgccatcctcgtcgccaccttccactacgagaagcgcccgcccgcgctcgaccagaacacgttcgcggactacaccgccaggacgtcgttcgagcggccgctgctgagcggggtggcgtacgcgcagcgggtggtgcacggcgacagggagagcttcgagcgccagcagggctggatcatcaagaccatgaagcacgagccgtctccggtgcaggatgagtacgcgccggtcgtttactcgcaggagaccgtctcctacattgaggggctcgacatgatgtccggcgaggaggaccgtgagaacattttgaggtcaagagcatcggggaaggcagttcttactagaccgttccggctcatgtcgaatcacctgggtgtcgtattgacttttcctgtctaccatgtcgatctttcttctgatgccaaggaggaggatcgtgttgctgccaccgcaggataccttgggggatcatttgatgtagaatcattagtggaaaatttgtttaggcagctagctggcaatcaggaattggtggtaaatgtttatgatgtcacaaacagttcgaaccctcttgtcatgtatggatcggaagtttctcttggcaacccctcaccatcgcacatctgcatgctagattttggcgatccattcagaaagcatcatatggtttgcagatacagaaacaagcctcagctcccatggtctgcaatatcttcgtcatctggtgtatttgtcatatgtatgcttgtggggtacatcgtgggtgccgcttggagtcgttatgataatgttaaggaagattgccggaaaatggaggagctgaaaaaacaggcagaagcagccgatgttgctaaatctcagttccttgcaactgtttctcatgagatcagaacgcccatgaatggagttctagggatgcttgatatgctgttagacactgacctaacgtcgacccagagggattttgcacaaacagctcaagtctgtggaaaggctttaatatcactaatcaatgaagtgcttgacagagcgaaaattgaagccggaaagttggatcttgagtctgtaccatttgacctgagatccatccttgatgatgtcatctcattattttcttcaaagtcaagagagaagggaattgagcttgctgtatatgtctctgaaagagttcctgaactcttgttgggtgatcctggaaggtttcggcagataattacaaatttagtgggcaactcaattaagttcacagaacggggacatatttttgtacaagttcatctggcagatcactcaaatcttgcaacagaatccaaagttgagtcagtggctaacgggatgaatggacataaagatgagaaaactgctgtagcaaccagtgtttctctcaacacactaagtggttttgaagctgctgatagccgaaatagttgggaaaacttcaagcttttgctttcttatgagaaaaatgagatgccctatgaaagtgtatctgataaagttactcttgtagtaagtgtggaagatacagggataggtataccattggatgcccaagccaaggtgttcactcctttcatgcaggccgatagttcgacttccaggacatatggtggtactggaattggattgagcatcagcaaatgtcttgttgaactaatgggtggtcagataaactttgttagccgaccacatgttgggagtacattcacatttactgcagctctccaaagatgtgacagaagcgctattggtgacagtaagcctgttatgttgcaccctcttccatccagtttcaaaggtttatctgcattattggttgatagaagaccagtaagagctactgtaactaagtatcacttgcaaaggctgggaattgcctgcgatgttgttgctaccattgaattggctcttggtgtgctgtccgggagaaatggcagttctctaaccagcacgaagcaaccatgcatgttattgattgagagtgattcatggggcttcaagattgatgtacctttacgatctcgactcctggagatgaagcagaatggtccacctggattgcccaaaactatccttctcgcagctgcagaatcgggcaaactcaaagcacactatgcagttgattctgtgatcacgaagcctctgaaagcaagcggacttgccgcttgtctattccaaacacttggcatcacacagtcaagcaacgagagacgcgacaactcaggttcccttcatgggttgctccttggcaagaacatattggtggttgatgacaacaaggtaaatcttagagtggctgctggcacattaaagaaattcggagcgaaggtggagtgcgtggagagtggaaaagatgctctcgccagcctacaagttccacataagttccatctttgtctcatggacattcagatgcccgaaatggatgggttcgaggccaccaagcaaataagggcaatggaagcgaaggcaaatgagcaggcagtcgcctgtgacgattcagatacggatggcgcgacaagggcggcaagatggcacctgcctgtccttgcaatgaccgccgatgtcatccaggccacccatgaggagtgcacaaagtacgggatggatgggtacgtcacgaagcccttcgaggagaagcagctcttccaggcgctgcagaagttcttggaccctggcatgtccagctaacacccaagtgctgcgttcgttgcaagtgaggcaccattctcct

SEQ ID NO: 12 - protein, 995 aa, Zea mays
MGVGGGGGGEAAAVSAPAPAEEAGKDAEDGGGWTLKAKLIAVAVLVWVLGALALGVFLHSYFRHAALRKAEEGLVSMCEERARMLQDQFAVSVNHVHALAILVATFHYEKRPPALDQNTFADYTARTSFERPLLSGVAYAQRVVHGDRESFERQQGWIIKTMKHEPSPVQDEYAPVVYSQETVSYIEGLDMMSGEEDRENILRSRASGKAVLTRPFRLMSNHLGVVLTFPVYHVDLSSDAKEEDRVAATAGYLGGSFDVESLVENLFRQLAGNQELVVNVYDVTNSSNPLVMYGSEVSLGNPSPSHICMLDFGDPFRKHHMVCRYRNKPQLPWSAISSSSGVFVICMLVGYIVGAAWSRYDNVKEDCRKMEELKKQAEAADVAKSQFLATVSHEIRTPMNGVLGMLDMLLDTDLTSTQRDFAQTAQVCGKALISLINEVLDRAKIEAGKLDLESVPFDLRSILDDVISLFSSKSREKGIELAVYVSERVPELLLGDPGRFRQIITNLVGNSIKFTERGHIFVQVHLADHSNLATESKVESVANGMNGHKDEKTAVATSVSLNTLSGFEAADSRNSWENFKLLLSYEKNEMPYESVSDKVTLVVSVEDTGIGIPLDAQAKVFTPFMQADSSTSRTYGGTGIGLSISKCLVELMGGQINFVSRPHVGSTFTFTAALQRCDRSAIGDSKPVMLHPLPSSFKGLSALLVDRRPVRATVTKYHLQRLGIACDVVATIELALGVLSGRNGSSLTSTKQPCMLLIESDSWGFKIDVPLRSRLLEMKQNGPPGLPKTILLAAAESGKLKAHYAVDSVITKPLKASGLAACLFQTLGITQSSNERRDNSGSLHGLLLGKNILVVDDNKVNLRVAAGTLKKFGAKVECVESGKDALASLQVPHKFHLCLMDIQMPEMDGFEATKQIRAMEAKANEQAVACDDSDTDGATRAARWHLPVLAMTADVIQATHEECTKYGMDGYVTKPFEEKQLFQALQKFLDPGMSS

SEQ ID NO: 13 - DNA, 3318 bp, Cucurbita maxima
  CDS (1)..(2946)
  3′UTR (2947)..(3318)
atgcaggtgagcgataactctgtgggtttgaagtggaatgagcaaatgggaacaacaaagaagggttacacatttgttcaagctaacagggcttggcttagaaagtatcttctgttctggattatggggatggcgtttatcagcatgttaatctataatggcatggatgctgatatcaaagtgaggaggaatgaagtgttggggagtatgtgtgagcagagggcaaggatgttgcaggatcaattcaatgttagtgttaaccatgttcatgccttggctgtccttgtttccacctttcattacttcaaaaacccttctgctattgatcaggaaacttttgcagaatacacagccagaactgcttttgaacggcctctactcagtggggtggcgtatgcacaaagagtgattcattcggagagggatatcttcgaaaagcaacacgggtggatgataagaacaatggaaaaggaaccttcgcccgatcgagatgaatatgcaccagtaatattttctcaagaaacagtctcgtatattgaatcgttggatatgatgtcaggagaggaggaccgggaaaatattttgagggctagagcaacaggaaaggctgtcttaacaagacccttcaggctgctgggttcccatcatcttggagttgttttgacatttcctgtttacaaattcaaattgccatccataccgactgaagaagaacggatagaagcaacagcaggctacgttggcggagcctttgatgttgagtcactcgtggagaacttgtttgggcagcttgcagggaatcaggccattttggtaaatgtatatgatgtcacgaactcttctgatcttctcgtgatgtatggtcatcaatatcaagatggtgacttgtcgctttcacatgagagcagccttgatttcggagatccattcaggaagcatttgatgatttgtagatatcagcagagggctcccacatcctggactgccctaactactgcattcttattcttcgtgatcggtttgttagttggatatattttgtatggtgcagcaactcacattgtgaaggttgaagatgattttcatgaaatgcaagtactgaaagttcgagcggaggctgccgatgtagcaaaatcccagtttcttgcaactgtttctcatgaaattaggacaccaatgaatggcatcctcggaatgcttgctctgcttctggatacagatctaagttccacacagaaggattatgctcaaactgcccaggcttgtggaaaggcattgatagcattaataaatgaggttcttgaccgggcaaaaattgaagctggaaagttagaactggaagcagttccattcgacattcgatcaatacttgatgacgtgctatctttattttccgagaagtccagacaaaagggtctggagctggcagtttttgtttctgataaagttccagaaattgtaattggagatcctggaagattcagacaaattataacaaatcttgtgggtaactctgttaagtttactgaaagaggacatatatttgttaaagtacacctagctgagaattcaaaagtctccatggactcggaatacgtcaacggaatatccgacagtggcttattcgtattggatggtcgtgaatttcaaactttgagtggacgcgaggcagccgatgatcagaacagttgggataacttcaagcatctaatcgctgacgacaacttccagtcgaatgccgcttcaaacaactcagcagttaccaacaagggttgtgatcatgttactttgatggtaagtgtggaggatactggaattgggatccttttacatgcccaaaatcgagttttcacacccttcatgcaagcagatagctcgacctcccgaaattatggagggactggtattggtttgagtatcagcaaatgtttagttgagttaatgggtggtcagatcaacttcataagccggcctcagattggaagcacgttttccttcactgctgtatttggaaaatgtaagaaaaactcgatgaatgatatgaaaaagcccaactctgaagaacttccccccagttttagaggaatgaaagcaatagtagttgatagcaaacatgtacgagcttctgtaaccaggtatcatttgaagagacttggtatcatagttgaagtcaccaatagcatcaacatggcagcttctttattcagagaaaatggatccacactgccaagaaacacaatccttccagatatgatcttagttgaaaaggacatactaaattctgatgaggaatgtgggatcattcatcatctgaactggaaaccgaacggtagttcggttaagtttccaaagctgatccttctcgctaccaatattgccactgctgaactagacaaggcaagagcagcaggttttgcagacaccgtgatcatgaagccgttgagggcgactatggtggctgcctgtcttcaacaagtactcggggttaagaatcagagacggccgaatggttctgctttcctccagagccttctctgtggcaagagaatcttaattgttgatgacaaccgagtaaaccgtcgggtcgctgcaggcgctctgaagaaatttggtgcagatgttgagtgtgcagatagcgggaaatctgcactgaagttgcttcagctaccgcataattttgatgcttgcttcatggatattcaaatgcctgaaatggatgggtttgaggcgactcgtcgtatcaggacaatggaggtcgaggcaaacaaaggaggattgtctgcaacagaaggcaaacggcctataccaatattagcaatgactgcagacgtgattcatgctacgtacgaagaatgcctgaaatgcggtatgaatggttacgtctcgaaaccctttgaagaagaaaatctatacaaggaagttgcccgatttttcaaaaaaccatagtccatcaaaagcttcatgaatgacaagaggtcatcagctgtagagctccttttggtgggttggaaagtaccagcaagttttgacaccattgctggtgctaactgtcctgttgctggtgaccgaggaaccgagttcaacgagggcgtcgggaattctcgactgccattgagactcgggtttgagctgctaccatttctaaccaactaaatattttattttgactagaatgtgagtacctacctgtatactacaccagaaatatccatccccaaatggatgtataattatggttgcaaggggaaggagctaaattgtaaatgctcatatttctaagacctcttctaaacatctttatagttggttggcttaggc

SEQ ID NO: 14 - protein, 981 aa, Cucurbita maxima
MQVSDNSVGLKWNEQMGTTKKGYTFVQANRAWLRKYLLFWIMGMAFISMLIYNGMDADIKVRRNEVLGSMCEQRARMLQDQFNVSVNHVHALAVLVSTFHYFKNPSAIDQETFAEYTARTAFERPLLSGVAYAQRVIHSERDIFEKQHGWMIRTMEKEPSPDRDEYAPVIFSQETVSYIESLDMMSGEEDRENILRARATGKAVLTRPFRLLGSHHLGVVLTFPVYKFKLPSIPTEEERIEATAGYVGGAFDVESLVENLFGQLAGNQAILVNVYDVTNSSDLLVMYGHQYQDGDLSLSHESSLDFGDPFRKHLMICRYQQRAPTSWTALTTAFLFFVIGLLVGYILYGAATHIVKVEDDFHEMQVLKVRAEAADVAKSQFLATVSHEIRTPMNGILGMLALLLDTDLSSTQKDYAQTAQACGKALIALINEVLDRAKIEAGKLELEAVPFDIRSILDDVLSLFSEKSRQKGLELAVFVSDKVPEIVIGDPGRFRQIITNLVGNSVKFTERGHIFVKVHLAENSKVSMDSEYVNGISDSGLFVLDGREFQTLSGREAADDQNSWDNFKHLIADDNFQSNAASNNSAVTNKGCDHVTLMVSVEDTGIGILLHAQNRVFTPFMQADSSTSRNYGGTGIGLSISKCLVELMGGQINFISRPQIGSTFSFTAVFGKCKKNSMNDMKKPNSEELPPSFRGMKAIVVDSKHVRASVTRYHLKRLGIIVEVTNSINMAASLFRENGSTLPRNTILPDMILVEKDILNSDEECGIIHHLNWKPNGSSVKFPKLILLATNIATAELDKARAAGFADTVIMKPLRATMVAACLQQVLGVKNQRRPNGSAFLQSLLCGKRILIVDDNRVNRRVAAGALKKFGADVECADSGKSALKLLQLPHNFDACFMDIQMPEMDGFEATRRIRTMEVEANKGGLSATEGKRPIPILAMTADVIHATYEECLKCGMNGYVSKPFEEENLYKEVARFFKKP

SEQ ID NO: 15 - protein, 993 aa, Lotus corniculatus
  Mutation (266)..(266): X is Xaa (any amino acid other than Leu)
MGLGFKMQQSHHPVALKLHEQAGSQRKFTFIQNFRNWFLPLLFVWFIVMAAFGACIYHKMDAETKVRRKEVLGSLCDQRARMLQDQFSVSVNHVHALAILVSTFHYYRNTSAIDQETFAEYTARTAFERPLMSGVAYAQRVVHSERERFEKQHGWVIKTMERVPSGVRDEYAAVIFAQETVSYLESIDMMSGEEDRENILRARATGKAVLTSPFRLLDSHHLGVVLTFPVYKSKLPPEPTTEEVIKAIAGYIGGSFDVESLVENLXGQLAGNQAILVKVYDITNSSDPLIMYGSQYEEGDMSLVHESKLDFGDPYRKHHMICRYHQQAPTNWIAYTTAFLFFVILCLVGYILYAAGTHIVKVEDDYNAMQDLKVKAEAADIAKSQFLATVSHEIRTPMNGILGMLGLLLRTELSSTQRDYAQTAQACGKALIALINEVLDRAKIEAGKLELEAVPFDLRSILDDVLSLFSEKSRHKGLELAVFVSDKVPDIVMGDPGRFRQIVTNLVGNSVKFTERGHIFVKVHLAEKRQCTMNGKCETFLNGGCDDVLHVSGSYNLKTLSGYEAADERNSWDNFKHHIADEEFFFDASVKKLASSESYEQVTLMVSVEDTGIGISFSAQDSIFMPFVQADSSTSRNYGGTGIGLSISKCLVELMGGQINFISRPQVGSTFSFTADFGTFKKNSTTDMKKLNFEDLPSSFRGLKAIVVDGKPVRAAVTRYHLKRLGIQAKVAISINKAVSLCGKNGSLTSALFQPDIIFVEKDSWVSGEDGGIFNAFKMPQMILLATNICNAEFDKAKAAGFSDTVIMKPLRASMLAACLQQVFGTGKTRQFGKDMSNGSSVRSLLCGKKILVVDDNLVNRRVAAGALKNFGADVKCAASGKAALEMLQYPHDFDACFMDIQMPEMDGFEATRRIRMMEREASEQLKSESGEENGKKSEFHMPILAMTADVIHATYDKCLNCGMDGYVSKPFEEENLYQAVAKFFKSKPASDS

SEQ ID NO: 16 - protein, 1003 aa, Medicago truncatula
  Mutation (267)..(267): X = Xaa (any amino aicd other than Leu)
MGLLLKMKMQNQHHPLASKLQEQTGNKRYTFIQAHRAWLLKLMFLWILLMALISRIIYSKMDVGTKVRRKEVLGSLCDQRARMLQDQFSVSVNHVHALAILVSTFHYYRNPSAIDQETFAEYTARTAFERPLLSGVAYAQRVVNSEREQFEKQHGVVIKTMEREASPVRDEYAPVIFAQETVSYLESIDMMSGEEDRENIMRARATGKAVLTSPFRLLGSHHLGVVLTFPVYKSKLPPNPTTEELIKATAGYVGGSFDVESLVENLXGQLAGHQAILVNVYDVTNSSDPLIMYGNQYEEGDVSLVHESKLDFGDPYRKHQMICRYHQKAPPNWTALSTAILFFVILLLIGYILYGAGNHIVKVEDDFHEMQELKVRAEAADVAKSQFLATVSHEIRTPMNGILGMLGLLLRTELNSTQRDYAQTAQACGKALIALINEVLDRAKIEAGKLELEAVPFDLRSILDDVLSLFSEKSRHKGLELAVFVSDKVPDIVMGDPGRFRQIVTNLVGNSVKFTERGHIFVKVHLSENRKPVTNGKHETYRNGGSEEVVHASGGYNLKTLSGYEAADERNNWDNFNHLIADEEFFCDASTKKVASNEFYEQVTLMVCVEDTGIGIPFSAQDRIFMPFVQADSSTSRNYGGTGIGLSISKCLVELMGGQINFISRPQVGSTFSFTADFGIFKKNPITEVKKVNYEDLPSSFRGLKAVVVDGKPVRAAVTRYHLKRLGIQVKVANAINKAVSLCGKNGASSTGLFQPDIIFVEKDSWVCGEDGIFSVRQLDWKQNGHIFKMPQMILLATNISNDEFDKAKSAGFSDTVIMKPLRASMVGACLQQVLGTGKKRQLGKEMPNGSTSVRSLLFGKKILVVDDNVVNRRVAAGALKNFGADVKCADSGKAALEMLQFPHKFDACFMDIQMPEMDGFEATRRIREMERTANEETNSECGERKSEFHLPILAMTADVIHATYEECLKCGMDGYVSKPFEEENLYQAVAKFFQTKPTSVDS

SEQ ID NO: 17 - protein, 1080 aa, Arabidopsis thaliana
  Mutation (356)..(356): X= Xaa (any amino acid other than Leucine)
MRRDFVYNNNAMFNPLTTHYSSDMNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLXGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKGIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKEAKEKTNLEWHLPILAMTADVIHATYEECLKSGMDGYVSKPFEEENLYKSVAKSFKPNPISPSS

SEQ ID NO: 18 - protein, 1013 aa, Oryza
  Mutation (266)..(266): X=Xaa (any amino acid other than Leu)
MGVGGGGGGGGGEAAAAVAVEGDEAGKGRRWWRVKVKLSTVAVVAWVLASAALWAGLHWRFRRAALHKAEEALVCMCEERARMLQDQFAVSVNHVHALAILVATFHYDKHPPALDQDTFAVYAARTSFERPLLSGVAYAQRVVHADRESFERQQGWIIKTMKHEPSPAQDEYAPVIYSQETISYIEGLDVMSGEEDRENILRARATGKAVLTRPFRLMSNHLGVVLTFPVYLVDLPNDTAVEDRVAATAGYLGGAFDVESLVENLXRQLAGNQELVVNVYDVTNHSNPLVMYGSEVPLGIPSPSHTYTLDFGDPLRKHQMVCRYRNKLHVSWSAITTPSGVFVICMLVGYIIYAAWSRYDNVKEDCRKMEALKKRAEAADIAKSQFLATVSHEIRTPMNGVLGMLDMLLDTELKSTQRDYAQTAQVCGKALISLINEVLDRAKIEAGKIDLESVPFDLRSILDDVISLFSSKSREKGIELAVYVSERVPEILLGDPGRFRQIITNLVGNSIKITIFTLSQFTERGHIFVQVHLADHSNLATEAKIEPVVNGMNGHKDEAIAIPTSGSHNTLSGFEAADSRNNWENFKLLLSYEKNEMPYESDSDKVTLVVSVEDTGIGIPLHAQGRVFTPFMQADSSTSRNYGGTGIGLSISKCLVEIMGGQINFVSRPLVGSTFTFTAVLRRCDKNAISDSKTVALHPLPSSFKGLSALLVDKRPVRATVTKYHLQRLGITSEVVGTIDPTFGVLSGRNGSSLTSIGKKQPCMLLIESDSWGPQMDVSLHARLQEMKQSDRIHVLPKVFLLSAAESDKVKKIHAVDSVIPKPLKASALAACLFQALGITQPSHEKRDDSGSLHGRDGSGSLHGLLLGKNILVVDDNKVNLRVAAGTLKKYGAKVECVESGKDALSLLQVPHKFDLCLMDIQMPEMDGFEATRQIRAMEGKANEQADDSESGSEIAAKTAKWHLPILAMTADVIQATHEECTKCGMDGYVSKPFEEKQLFQAVQKFLGPCVSS

SEQ ID NO: 19 - protein, 995 aa, Zea mays
  Mutation (267)..(267): X= Xaa (any amino acid other than Leu)
MGVGGGGGGEAAAVSAPAPAEEAGKDAEDGGGWTLKAKLIAVAVLVWVLGALALGVFLHSYFRHAALRKAEEGLVSMCEERARMLQDQFAVSVNHVHALAILVATFHYEKRPPALDQNTFADYTARTSFERPLLSGVAYAQRVVHGDRESFERQQGWIIKTMKHEPSPVQDEYAPVVYSQETVSYIEGLDMMSGEEDRENILRSRASGKAVLTRPFRLMSNHLGVVLTFPVYHVDLSSDAKEEDRVAATAGYLGGSFDVESLVENLXRQLAGNQELVVNVYDVTNSSNPLVMYGSEVSLGNPSPSHICMLDFGDPFRKHHMVCRYRNKPQLPWSAISSSSGVFVICMLVGYIVGAAWSRYDNVKEDCRKMEELKKQAEAADVAKSQFLATVSHEIRTPMNGVLGMLDMLLDTDLTSTQRDFAQTAQVCGKALISLINEVLDRAKIEAGKLDLESVPFDLRSILDDVISLFSSKSREKGIELAVYVSERVPELLLGDPGRFRQIITNLVGNSIKFTERGHIFVQVHLADHSNLATESKVESVANGMNGHKDEKTAVATSVSLNTLSGFEAADSRNSWENFKLLLSYEKNEMPYESVSDKVTLVVSVEDTGIGIPLDAQAKVFTPFMQADSSTSRTYGGTGIGLSISKCLVELMGGQINFVSRPHVGSTFTFTAALQRCDRSAIGDSKPVMLHPLPSSFKGLSALLVDRRPVRATVTKYHLQRLGIACDVVATIELALGVLSGRNGSSLTSTKQPCMLLIESDSWGFKIDVPLRSRLLEMKQNGPPGLPKTILLAAAESGKLKAHYAVDSVITKPLKASGLAACLFQTLGITQSSNERRDNSGSLHGLLLGKNILVVDDNKVNLRVAAGTLKKFGAKVECVESGKDALASLQVPHKFHLCLMDIQMPEMDGFEATKQIRAMEAKANEQAVACDDSDTDGATRAARWHLPVLAMTADVIQATHEECTKYGMDGYVTKPFEEKQLFQALQKFLDPGMSS

SEQ ID NO: 20 - protein, 981 aa, Cucurbita maxima
  Mutation (261)..(261): X= Xaa (any amino acid other than Leu)
MQVSDNSVGLKWNEQMGTTKKGYTFVQANRAWLRKYLLFWIMGMAFISMLIYNGMDADIKVRRNEVLGSMCEQRARMLQDQFNVSVNHVHALAVLVSTFHYFKNPSAIDQETFAEYTARTAFERPLLSGVAYAQRVIHSERDIFEKQHGWMIRTMEKEPSPDRDEYAPVIFSQETVSYIESLDMMSGEEDRENILRARATGKAVLTRPFRLLGSHHLGVVLTFPVYKFKLPSIPTEEERIEATAGYVGGAFDVESLVENLXGQLAGNQAILVNVYDVTNSSDLLVMYGHQYQDGDLSLSHESSLDFGDPFRKHLMICRYQQRAPTSWTALTTAFLFFVIGLLVGYILYGAATHIVKVEDDFHEMQVLKVRAEAADVAKSQFLATVSHEIRTPMNGILGMLALLLDTDLSSTQKDYAQTAQACGKALIALINEVLDRAKIEAGKLELEAVPFDIRSILDDVLSLFSEKSRQKGLELAVFVSDKVPEIVIGDPGRFRQIITNLVGNSVKFTERGHIFVKVHLAENSKVSMDSEYVNGISDSGLFVLDGREFQTLSGREAADDQNSWDNFKHLIADDNFQSNAASNNSAVTNKGCDHVTLMVSVEDTGIGILLHAQNRVFTPFMQADSSTSRNYGGTGIGLSISKCLVELMGGQINFISRPQIGSTFSFTAVFGKCKKNSMNDMKKPNSEELPPSFRGMKAIVVDSKHVRASVTRYHLKRLGIIVEVTNSINMAASLFRENGSTLPRNTILPDMILVEKDILNSDEECGIIHHLNWKPNGSSVKFPKLILLATNIATAELDKARAAGFADTVIMKPLRATMVAACLQQVLGVKNQRRPNGSAFLQSLLCGKRILIVDDNRVNRRVAAGALKKFGADVECADSGKSALKLLQLPHNFDACFMDIQMPEMDGFEATRRIRTMEVEANKGGLSATEGKRPIPILAMTADVIHATYEECLKCGMNGYVSKPFEEENLYKEVARFFKKP

SEQ ID NO: 21 - protein, 1013 aa, Oryza sativa
  PEPTIDE (1)..(1013): Oryza sativum wild-type protein
MGVGGGGGGGGGEAAAAVAVEGDEAGKGRRWWRVKVKLSTVAVVAWVLASAALWAGLHWRFRRAALHKAEEALVCMCEERARMLQDQFAVSVNHVHALAILVATFHYDKHPPALDQDTFAVYAARTSFERPLLSGVAYAQRVVHADRESFERQQGWIIKTMKHEPSPAQDEYAPVIYSQETISYIEGLDVMSGEEDRENILRARATGKAVLTRPFRLMSNHLGVVLTFPVYLVDLPNDTAVEDRVAATAGYLGGAFDVESLVENLLRQLAGNQELVVNVYDVTNHSNPLVMYGSEVPLGIPSPSHTYTLDFGDPLRKHQMVCRYRNKLHVSWSAITTPSGVFVICMLVGYIIYAAWSRYDNVKEDCRKMEALKKRAEAADIAKSQFLATVSHEIRTPMNGVLGMLDMLLDTELKSTQRDYAQTAQVCGKALISLINEVLDRAKIEAGKIDLESVPFDLRSILDDVISLFSSKSREKGIELAVYVSERVPEILLGDPGRFRQIITNLVGNSIKITIFTLSQFTERGHIFVQVHLADHSNLATEAKIEPVVNGMNGHKDEAIAIPTSGSHNTLSGFEAADSRNNWENFKLLLSYEKNEMPYESDSDKVTLVVSVEDTGIGIPLHAQGRVFTPFMQADSSTSRNYGGTGIGLSISKCLVEIMGGQINFVSRPLVGSTFTFTAVLRRCDKNAISDSKTVALHPLPSSFKGLSALLVDKRPVRATVTKYHLQRLGITSEVVGTIDPTFGVLSGRNGSSLTSIGKKQPCMLLIESDSWGPQMDVSLHARLQEMKQSDRIHVLPKVFLLSAAESDKVKKIHAVDSVIPKPLKASALAACLFQALGITQPSHEKRDDSGSLHGRDGSGSLHGLLLGKNILVVDDNKVNLRVAAGTLKKYGAKVECVESGKDALSLLQVPHKFDLCLMDIQMPEMDGFEATRQIRAMEGKANEQADDSESGSEIAAKTAKWHLPILAMTADVIQATHEECTKCGMDGYVSKPFEEKQLFQAVQKFLGPCVSS

SEQ ID NO: 22 - protein, 995 aa, Zea mays
  PEPTIDE (1)..(995): Zea mays wild type protein (histidine kinase)
MGVGGGGGGEAAAVSAPAPAEEAGKDAEDGGGWTLKAKLIAVAVLVWVLGALALGVFLHSYFRHAALRKAEEGLVSMCEERARMLQDQFAVSVNHVHALAILVATFHYEKRPPALDQNTFADYTARTSFERPLLSGVAYAQRVVHGDRESFERQQGWIIKTMKHEPSPVQDEYAPVVYSQETVSYIEGLDMMSGEEDRENILRSRASGKAVLTRPFRLMSNHLGVVLTFPVYHVDLSSDAKEEDRVAATAGYLGGSFDVESLVENLLRQLAGNQELVVNVYDVTNSSNPLVMYGSEVSLGNPSPSHICMLDFGDPFRKHHMVCRYRNKPQLPWSAISSSSGVFVICMLVGYIVGAAWSRYDNVKEDCRKMEELKKQAEAADVAKSQFLATVSHEIRTPMNGVLGMLDMLLDTDLTSTQRDFAQTAQVCGKALISLINEVLDRAKIEAGKLDLESVPFDLRSILDDVISLFSSKSREKGIELAVYVSERVPELLLGDPGRFRQIITNLVGNSIKFTERGHIFVQVHLADHSNLATESKVESVANGMNGHKDEKTAVATSVSLNTLSGFEAADSRNSWENFKLLLSYEKNEMPYESVSDKVTLVVSVEDTGIGIPLDAQAKVFTPFMQADSSTSRTYGGTGIGLSISKCLVELMGGQINFVSRPHVGSTFTFTAALQRCDRSAIGDSKPVMLHPLPSSFKGLSALLVDRRPVRATVTKYHLQRLGIACDVVATIELALGVLSGRNGSSLTSTKQPCMLLIESDSWGFKIDVPLRSRLLEMKQNGPPGLPKTILLAAAESGKLKAHYAVDSVITKPLKASGLAACLFQTLGITQSSNERRDNSGSLHGLLLGKNILVVDDNKVNLRVAAGTLKKFGAKVECVESGKDALASLQVPHKFHLCLMDIQMPEMDGFEATKQIRAMEAKANEQAVACDDSDTDGATRAARWHLPVLAMTADVIQATHEECTKYGMDGYVTKPFEEKQLFQALQKFLDPGMSS

SEQ ID NO: 23 - protein, 1080 aa, Arabidopsis thaliana
  PEPTIDE (1)..(1080): Arabidopsis thaliana wild type protein (histidine kinase)
MRRDFVYNNNAMFNPLTTHYSSDMNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKGIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKEAKEKTNLEWHLPILAMTADVIHATYEECLKSGMDGYVSKPFEEENLYKSVAKSFKPNPISPSS

SEQ ID NO: 24 - protein, 1003 aa, Medicago truncatula
  PEPTIDE (1)..(1003): Medicago trunculata wild type protein (histidine kinase)
MGLLLKMKMQNQHHPLASKLQEQTGNKRYTFIQAHRAWLLKLMFLWILLMALISRIIYSKMDVGTKVRRKEVLGSLCDQRARMLQDQFSVSVNHVHALAILVSTFHYYRNPSAIDQETFAEYTARTAFERPLLSGVAYAQRVVNSEREQFEKQHGVVIKTMEREASPVRDEYAPVIFAQETVSYLESIDMMSGEEDRENIMRARATGKAVLTSPFRLLGSHHLGVVLTFPVYKSKLPPNPTTEELIKATAGYVGGSFDVESLVENLLGQLAGHQAILVNVYDVTNSSDPLIMYGNQYEEGDVSLVHESKLDFGDPYRKHQMICRYHQKAPPNWTALSTAILFFVILLLIGYILYGAGNHIVKVEDDFHEMQELKVRAEAADVAKSQFLATVSHEIRTPMNGILGMLGLLLRTELNSTQRDYAQTAQACGKALIALINEVLDRAKIEAGKLELEAVPFDLRSILDDVLSLFSEKSRHKGLELAVFVSDKVPDIVMGDPGRFRQIVTNLVGNSVKFTERGHIFVKVHLSENRKPVTNGKHETYRNGGSEEVVHASGGYNLKTLSGYEAADERNNWDNFNHLIADEEFFCDASTKKVASNEFYEQVTLMVCVEDTGIGIPFSAQDRIFMPFVQADSSTSRNYGGTGIGLSISKCLVELMGGQINFISRPQVGSTFSFTADFGIFKKNPITEVKKVNYEDLPSSFRGLKAVVVDGKPVRAAVTRYHLKRLGIQVKVANAINKAVSLCGKNGASSTGLFQPDIIFVEKDSWVCGEDGIFSVRQLDWKQNGHIFKMPQMILLATNISNDEFDKAKSAGFSDTVIMKPLRASMVGACLQQVLGTGKKRQLGKEMPNGSTSVRSLLFGKKILVVDDNVVNRRVAAGALKNFGADVKCADSGKAALEMLQFPHKFDACFMDIQMPEMDGFEATRRIREMERTANEETNSECGERKSEFHLPILAMTADVIHATYEECLKCGMDGYVSKPFEEENLYQAVAKFFQTKPTSVDS

(19) United States
(12) Patent Application Publication
(10) Pub. No.: US 2010/0031388 A1
(43) Pub. Date: Feb. 4, 2010

(54) MUTANT HISTIDINE KINASE THAT CONFERS SPONTANEOUS NODULATION IN PLANTS

(75) Inventors: Leila Tirichine, Risskov (DK); Jens Stougaard, Aarhus (DK); Niels Norgaard Sandal, Tilst (DK); Lene H. Madson, Risskov (DK); Elena Simona Radutoiu, Aarhus (DK)

Correspondence Address: DARBY & DARBY P.C., P.O. BOX 770, Church Street Station, New York, NY, 10008-0770, US

(73) Assignee: Plant Biosciences Ltd., Norfolk (GB)

(21) Appl. No.: 12/374,413

(22) PCT Filed: Jul. 21, 2006
(86) PCT No.: PCT/DK06/50031

§ 371 Date: Aug. 19, 2009

(51) Int. Cl. A01H 1/00; C07H 21/00; C12N 9/12; A01H 5/00; A01H 5/10; C12N 15/82

(52) U.S. Cl. 800/278; 536/23.2; 435/194; 800/295; 800/298; 800/320; 800/320.2; 800/320.1; 800/320.3

(57) ABSTRACT

Formation of nitrogen fixing root nodules in legumes is induced by perception of lipochitin-oligosaccharide signal molecules secreted by compatible Rhizobium bacteria, which triggers a common symbiotic pathway. The present invention provides a spontaneous nodule formation (snf2) mutant, in which the formation of symbiotic nodules is spontaneous, leading to nodule development in the absence as well as in the presence of Rhizobium bacteria and/or exogenous rhizobial signals. The invention further provides an isolated DNA sequence encoding a mutant cytokinin-independent histidine kinase whose activity results in this ‘gain of function’ dominant phenotype of spontaneous nodule formation. Furthermore the snf2 gene is shown to confer a phenotype, characterised by regulated organogenesis of spontaneous nodules, to plants having a nodulation deficient genetic background. A gene of the invention, that confers this spontaneous nodulation phenotype, has 15 utility for the transfer and establishment of nitrogen fixing capability in non-nodulating plants, and thereby reducing the nitrogen fertiliser dependence of non-nodulating crop plants.

### BACKGROUND OF THE INVENTION

The growth of agricultural crops is generally limited by the availability of nitrogen, and at least 50% of global requirement is met by the application of synthetic fertilisers in the form of ammonia, nitrate or urea. However, there is a growing need to exploit one of the most important natural sources of nitrogen for agriculture, namely biological nitrogen fixation.

The primary source of biological nitrogen fixation are Rhizobium or Rhizobia spp and the actinobacterium Frankia spp, which are a small group of prokaryotes that produce nitrogenases, and form endosymbiotic associations with plants conferring the ability to fix nitrogen. Although many plants can associate with nitrogen-fixing bacteria, only a few plants, all members of the Rosid I Clade, form an endosymbiotic association with Rhizobia spp and Frankia spp., which are unique in that most of the nitrogen is transferred to and assimilated by the host plant. The Leguminosae plant family, which includes soybean, bean, pea, peanut, chickpea, cowpea, lentil, pigeonpea, alfalfa and clover, are the most agronomically important members of this small group of nitrogen-fixing plants. Biological nitrogen fixation via the endosymbiotic association reduces the need for expensive nitrogen fertilizers in legume crops and is an important feature of sustainable agriculture. Legumes can also utilize nitrogen available in the soil, such that when levels of soil nitrate are high, nodule formation is suppressed and the plant shifts from nitrogen metabolism to growth on nitrate (Wopereis et al., 2000).

Rhizobium-legume symbiosis involves the interaction of a set of plant and bacterial genes in a complex process leading to the initiation and development of root nodules. Organogenesis of nodules is triggered by the rhizobial microsymbiont, but the legume host plant encodes the developmental program responsible for building the nodule tissues and for regulating the process. Low molecular weight lipo-chitin-oligosaccharides (Nod-factors), synthesized and secreted by Rhizobia, are major signal molecules that trigger this process. The major Nod-factor secreted by the Mesorhizobium loti microsymbiont of Lotus is a pentameric N-acetylglucosamine carrying a cis-vaccenic acid and a carbamoyl group at the non-reducing terminal residue together with a 4-O-acetylfucose at the reducing terminal residue. Perception of Nod-factor in Lotus is mediated by NFR1 and NFR5 receptor kinases (Radutoiu et al., 2003 Nature 425: 585-592; Madsen et al., 2003 Nature 425: 637-640), that together with an LRR receptor-kinase gene, SymRK, communicate with a common signal transduction pathway, shared with mycorrhizal symbiosis (Oldroyd and Downie, 2004 Mol. Cell. Biology 5: 566-576). This common pathway is encoded by seven genes, SymRK, Castor, Pollux, Nup133, CCaMK [Sym15], Sym6 and Sym24. Analysis of mutants has shown that NFR1/NFR5 receptor(s), SymRK encoded LRR protein kinase, CASTOR/POLLUX cation channel(s) and nucleoporin133 are required for the induction of calcium spiking, one of the earliest physiological responses detectable in root hairs exposed to purified Nod-factor.

To establish symbiosis, Rhizobia gain access to single plant cells by endocytosis where they are installed in symbiosomes surrounded by a peribacteroid membrane. In Lotus, infection occurs via an infection thread that takes the bacteria through root hairs into the root cortex and distributes them to cells, which become infected symbiosome containing nitrogen-fixing cells. In response to attached bacteria, root hairs deform and curl, setting up a pocket that provides a site for infection thread initiation (Geurts et al., 2005 Curr. Opinion Plant Biol., 8: 346-352). Infection threads are plant-derived structures originating from plasma membrane invagination, accompanied by external deposition of cell wall material. In advance of the inward progressing intracellular thread, root cortical cells dedifferentiate and re-enter the cell cycle to initiate the nodule primordium. Later in the process, pattern formation and cell differentiation specify tissue and cell types including the infected cells that endocytose Rhizobia. In the mature functional nodule, peripheral vascular bundles are connected to the root vasculature and the main tissues/cell types can be distinguished (Pawlowski and Bisseling, 1996, Plant Cell 8: 1899-1913).

Analysis of a group of nodulation mutants, including some that fail to show calcium oscillations in response to Nod-factor signals, has revealed that in addition to the lack of nodulation, these mutants are unable to form endosymbioses with arbuscular mycorrhizal fungi. This implies that a common symbiotic signal transduction pathway is shared by two types of endosymbiotic relationships, namely root nodule symbiosis, which is largely restricted to the legume family, and arbuscular mycorrhizal symbiosis, which is common to the majority of land plant species. This suggests that there may be a few key genes which dispose legumes to engage in nodulation, and which are missing from crop plants such as cereals. The identification of these key genes, which encode functions which are indispensable for establishing a nitrogen fixing system in legumes, and their transfer and expression in non-nodulating plants, has long been a goal of molecular plant breeders. This could have a significant agronomic impact on the cultivation of cereals such as rice, where production of two harvests a year may require fertilisation with up to 400 kg nitrogen per hectare.

Root nodule symbiosis depends on a successful interaction between the plant host and its cognate symbiont that includes the step of nod factor recognition by the host plant. The identification of genes that regulate nodulation in Legumes would provide the tools to optimise and modify this process to the benefit of agriculture.

In summary, there is a need to improve nodule formation capability and nitrogen fixation properties in legume crops, as well as to transfer this pathway into non-nodulating crops in order to meet the nutritional needs of a growing global population, while minimising the future use of nitrogen fertilisers and their associated negative environmental impact.

### SUMMARY OF THE INVENTION

A first embodiment of the invention is a DNA molecule encoding a mutant histidine kinase polypeptide comprising an amino acid sequence selected from among: SEQ ID NO: 6, 9, 12, 14-20, wherein the amino acid residue corresponding to Xaa is selected from among isoleucine, serine, threonine, valine, methionine, alanine, phenylalanine, tyrosine, tryptophan, arginine, lysine, glycine, histidine, aspartate, asparagine, glutamate, glutamine, proline and cysteine,

an orthologue of (a) and a truncation of (a) or (b), capable of inducing spontaneous nodule formation in a plant.

A second embodiment of the invention is a mutant histidine kinase polypeptide consisting of an amino acid sequence selected from among: (a) SEQ ID NO: 6, 9, 12, 14-20, wherein the amino acid residue corresponding to Xaa is selected from among isoleucine, serine, threonine, valine, methionine, alanine, phenylalanine, tyrosine, tryptophan, arginine, lysine, glycine, histidine, aspartate, asparagine, glutamate, glutamine, proline and cysteine; (b) a cytokinin independent allelic variant of (a); (c) a cytokinin independent orthologue of (a); and (d) a truncation of (a), (b) or (c).

A further embodiment of the invention is a genetically modified plant characterised by having a nucleotide sequence encoding a polypeptide comprising an mutant histidine kinase consisting of an amino acid sequence selected from among: (a) SEQ ID NO: 6, 9, 12, 14-20, wherein the amino acid residue corresponding to Xaa is selected from among isoleucine, serine, threonine, valine, methionine, alanine, phenylalanine, tyrosine, tryptophan, arginine, lysine, glycine, histidine, aspartate, asparagine, glutamate, glutamine, proline and cysteine; (b) an cytokinin independent allelic variant of (a); (c) a cytokinin independent orthologue of (a); and (d) a truncation of (a), (b) or (c), wherein said plant is capable of spontaneous nodule formation. The invention is further directed to the use of a nucleic acid molecule encoding a mutant histidine kinase consisting of an amino acid sequence selected from among: (a) SEQ ID NO: 7, 8, 9, 10, 11, 15, 26 and 27, wherein the amino acid residue corresponding to Xaa is selected from among isoleucine, serine, threonine, valine, methionine, alanine, phenylalanine, tyrosine, tryptophan, arginine, lysine, glycine, histidine, aspartate, asparagine, glutamate, glutamine, proline and cysteine; (b) an orthologue of (a); and (c) a truncation of (a) or (b), as a transgene to produce the genetically modified plant of the invention capable of spontaneous nodulation according to its various embodiments.

The invention is further directed a method of producing a genetically modified plant according to the invention in its various embodiments, characterised by introducing a gene cassette comprising said nucleotide sequence encoding said polypeptide and selecting a transgenic plant and its progeny expressing said polypeptide.

The invention further includes a genetically modified plant produced according to a process of DNA mutagenesis and selecting a plant capable of spontaneous nodule formation, or by a method of transformation with a transgene encoding a mutant histidine kinase of the invention.

The invention further includes a seed or a crop obtained from the genetically modified plant of the invention. Furthermore the invention is directed to the use of a genetically modified plant according to the invention in a breeding program, and a plant selected in the breeding program comprising a nucleotide sequence encoding a polypeptide comprising a mutant histidine kinase consisting of an amino acid sequence selected from among: (a) SEQ ID NO: 6, 9, 12, 14-20 wherein the amino acid residue corresponding to Xaa is selected from among isoleucine, serine, threonine, valine, methionine, alanine, phenylalanine, tyrosine, tryptophan, arginine, lysine, glycine, histidine, aspartate, asparagine, glutamate, glutamine, proline and cysteine; (b) an cytokinin independent allelic variant of (a); (c) a cytokinin independent orthologue of (a), and (d) a truncation of (a), (b) or (c), wherein said plant is capable of spontaneous nodule formation.

### BRIEF DESCRIPTION OF THE DRAWINGS

FIG. 1. Phenotypic characterisation of the snf2 mutant.

(A) wild type rhizobia induced root nodule (B) spontaneous root nodule on snf2 root 5 weeks post germination. Arrowheads indicate nodules. Transverse section of (C) wild type and (D) snf2 root at time zero and (E) wild type and (F) snf2 root after 6 days of growth on B5 hormone-free medium. Arrowhead indicates dividing cells in the pericycle. Arrows indicate xylem cells. (G) and (H) callus growth from hypocotyls of wild type and snf2 on different concentrations of auxin and cytokinin. The calli were photographed after 21 days of growth at 26° C. (I) and (J) root segments of wild type and snf2 incubated for three weeks on hormone free media. Scale bars: (C); (D); (E); (F) 50 μm.

FIG. 2. Map based cloning of the Lhk1 gene

A: Cartoon of Lotus japonicus chromosome IV with expanded genetic map of snf2 region (Lhk1 locus) below, comprising 6 BAC/TAC clones (underlined in bold). Vertical lines indicate microsatellites or single nucleotide polymorphism markers. Number of recombinant plants obtained for a microsatellite (TM) marker is indicated in bold below the marker.

B: Exon-intron structure of the Lhk1 gene, where the 11 exons of Lhk1 are indicated by open boxes. The C→T transition in exon 4 of the mutant snf2 allele, (indicated with a black arrowhead) encodes a mutant LHK1 polypeptide comprising a substitution of F266 for L266.

FIG. 3. Transformation of snf2 allele into wild type hairy roots leads to controlled organogenesis and spontaneous nodulation phenotype. Lotus japonicus wild type plants were transformed with Agrobacterium rhizogenes carrying the snf2 gene. Spontaneous nodules on hairy roots are identified by white arrowheads. The main root is identified by a white arrow.

FIG. 4. Structure of the Lotus LHK1 protein.

(A) Schematic representation of the LHK1 protein domains. (B) The amino acid sequence of LHK1 arranged in protein domains. The predicted extracellular receptor domain is given in italics and the CHASE domain within the extracellular receptor domain is underlined; the histidine kinase domain is given in bold and underlined; the His Kinase ATPase domain is given in bold; and the receiver domain is in bold and italics. The asterisk in the CHASE domain marks the position of the amino acid substitution in the snf2 allele.

FIG. 5. In vivo assays of receptor mediated cytokinin signalling.

(A) Plate assay of β-galactosidase activity expressed from a cps::lacZ reporter gene in E. coli. An E. coli SRC122 strain carrying the cps::lacZ reporter, transformed with a plasmid construct comprising the snf2 cDNA or wild type cDNA, was grown on plates in the absence of cytokinin or in presence of four different cytokinins. The blue color shows β-galactosidase conversion of X-Gal substrate.

(B) Cytokinin induction of β-galactosidase activity in liquid cultures of SRC122 cps::lacZ transformed with either the snf2 cDNA or wild type cDNA. T-z: Trans-zeatin.

(C) Working model for the functional role of Lhk1 in nodulation. Recognition of a correctly decorated rhizobial Nod-factor by NFR1 and NFR5 induces signal transduction through the common pathway, including calcium spiking and interpretation of calcium oscillation by the CCaMK protein. A localised increase in cytokinin biosynthesis perceived by the LHK1 receptor then leads to cell dedifferentiation and activation of the cell cycle. snf2 is constitutively active but still requires Nin and Sym35 genes for nodule organogenesis.

FIG. 6. Effect of phytohormone treatment on callus growth of root explants. Callus growth of wild type L. japonicus ecotype Gifu (A) and (B) snf2 root segments on different concentrations of auxin and cytokinin. The calli were photographed after 21 days of growth at 26° C.

FIG. 7. Expression of the Lhk1 gene in organs and the Lhk1, Lrr5 and nin in response to cytokinin. (A) Expression of Lhk1 in different organs. (B) Expression of Lrr5 gene in wild type and snf2 root explants incubated on B5 agar plates with or without 0.5 μg/ml of BAP for 10 days. (C), (D), (E) Expression of Lrr5, Lhk1 and Nin, genes in intact wild type and snf2 roots in response to cytokinin. (F) Expression of Nin gene in wild type and snf2 root explants incubated on B5 agar plates with or without 0.5 μg/ml of BAP for 10 days.

FIG. 8: Effect of cytokinin on shoot and root length of L. japonicus wild type and snf2-2 mutant. Plants were grown on ¼ B&D containing different concentrations of BAP. Shoot and root length were measured after 3 weeks of growth. (A) shoot length (B) root length.

FIG. 9. Nodulation phenotype of snf2 and the double mutant snf2 har1 in absence of rhizobia. (A) snf2 mutant and (B) snf2 har1 double mutant 5 weeks post germination

### DETAILED DESCRIPTION OF THE INVENTION

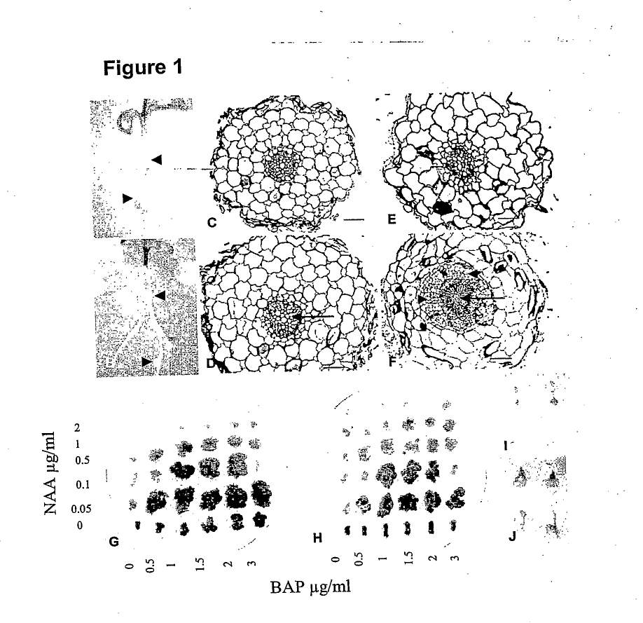

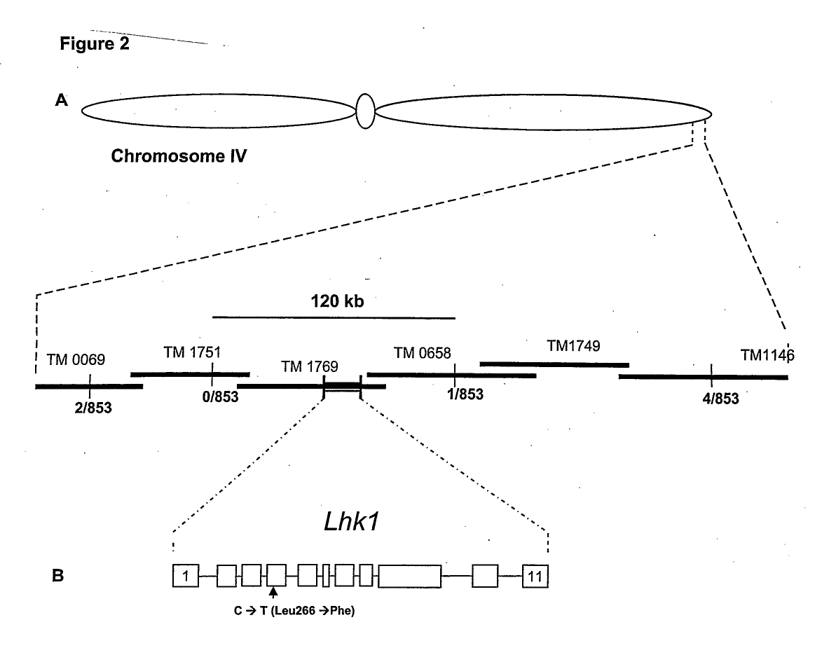

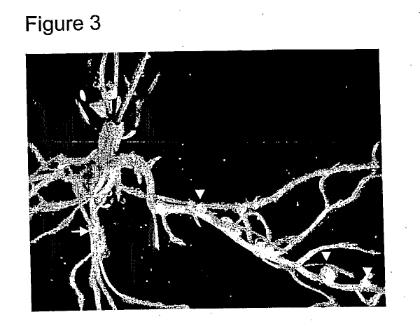

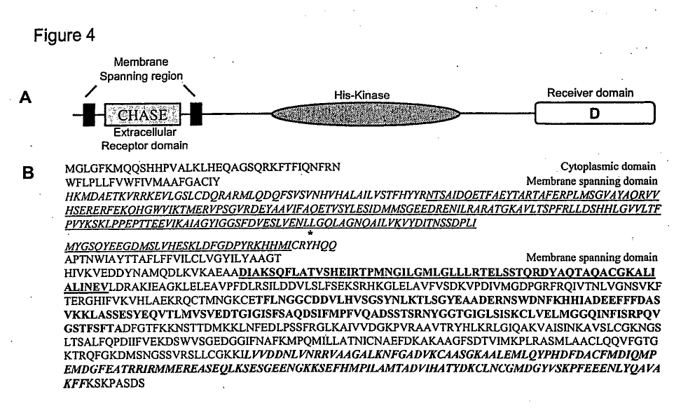

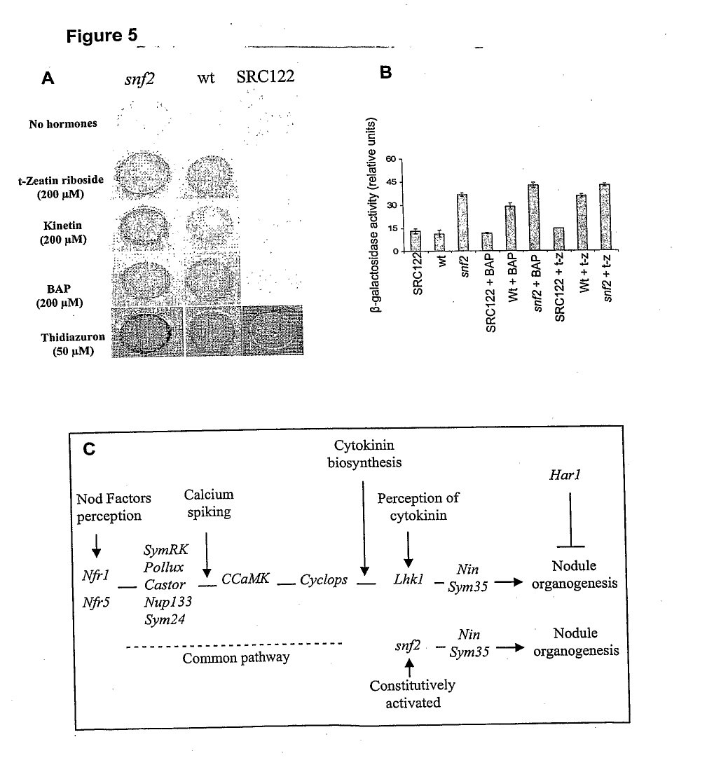

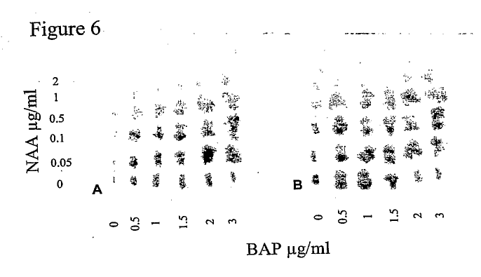

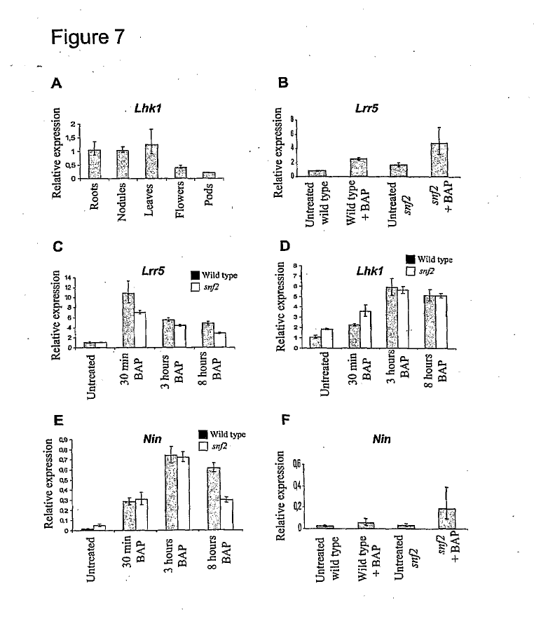

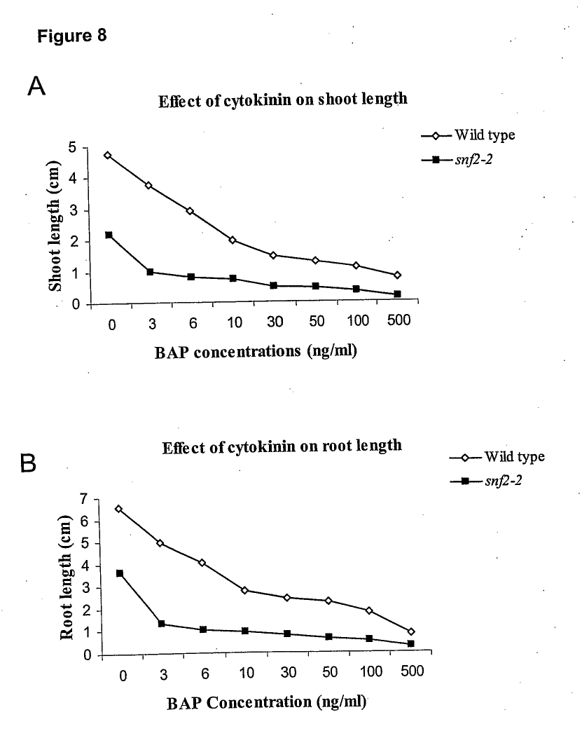

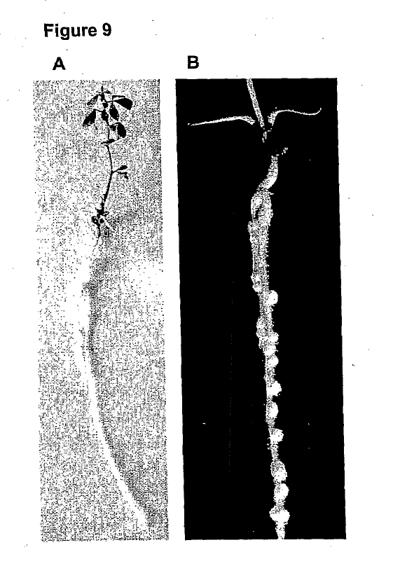

The present invention provides an isolated gene encoding a mutant polypeptide, whose expression in plants confers a spontaneous nodulation phenotype. More specifically the gene was isolated from snf2 spontaneous nodulation mutants of Lotus japonicus that develop white rhizobia free nodules in absence of the M. loti microsymbiont, while wild type Lotus plants are only nodulated following induction with its cognate nod-factors or rhizobial symbiont (FIG. 1a, b). Detailed histological analyses of nodule sections demonstrate that snf2 spontaneous nodules are genuine nodules with an ontogeny and physiology similar to rhizobial induced nodules. Thus, spontaneous root nodules formed on snf2 mutants originate from cortical cells and have a peripheral vasculature characteristic for wild-type nodules, although they are devoid of infection threads and rhizobia. The snf2 allele is monogenic dominant and inoculation of snf2 mutants with M. loti results in development of normal nitrogen-fixing root nodules consistent with the presence of a gain-of-function mutation in this allele.

The snf2 allele maps to a position on chromosome 4 approximately 1 cM from the end of the long arm (FIG. 2). The gene, corresponding to the snf2 allele, encodes a mutant form of a Lotus Histidine Kinase (LHK1) that is a homolog of Arabidopsis histidine kinase genes (AHK) encoding cytokinin receptor proteins. A comparison of the wild type Lotus histidine kinase gene (Lhk1) [SEQ ID NO:1] with the corresponding gene of the snf2 allele [SEQ ID NO:4] reveals a single nucleotide transition (C to T) in the coding sequence of the snf2 allele that results in the substitution of a conserved leucine [L266 in Lhk1; SEQ ID NO:3] by a phenylalanine (F266 in snf2 allele of Lhk1; SEQ ID NO:6) in the encoded polypeptide. The invention further provides isolated cDNAs corresponding to a transcript of the wild type Lhk1 gene [SEQ ID NO:2] and that of the snf2 allele [SEQ ID NO:5]. Alignment of the Lhk1 genomic and cDNA sequences or the respective snf2 allele gene and cDNA sequences, defines a primary structure of Lhk1 and its snf2 allele as consisting of 11 exons (FIG. 2).

The invention further provides isolated wild type and mutant cytokinin receptor proteins (LHK1) encoded by the Lhk1 gene and its snf allele respectively, which comprise 993 amino acids with a predicted mass of 110 kD (FIG. 4). Both proteins comprise two membrane spanning segments at the N-terminus, located between amino acids 37 and 57 and between amino acids 328 and 357. Located between these segments are motifs characteristic of cyclases/histidine kinases associated sensory extracellular (CHASE) domain. This predicted extracellular domain is followed by a putative intracellular histidine kinase between amino acids 379 and 693 and a receiver domain between amino acids 852 and 985. These domains are characteristic for two-component regulatory systems operating through a phospho-relay. The phenylalanine 266 in the snf2 allele, substituted for leucine 266, is localised in a conserved motif shared among the extracellular CHASE domains of histidine kinase receptors (FIG. 4). The functional properties of the histidine kinase (cytokinin receptor protein) encoded by the snf2 allele include cytokinin-independent activity, such that the snf2 allele, in contrast to the wild type Lhk1 allele, can induce nodulation in the absence of cytokinin signalling.

Accordingly, the present invention provides an isolated mutant histidine kinase protein (mutant cytokinin receptor protein) capable of inducing spontaneous nodulation when expressed in a plant, wherein the amino acid residue corresponding to L266 in wild type LHK1 histidine kinase encoded by the Lotus japonicus Lhk1 gene is substituted by an amino acid selected from isoleucine, serine, threonine, valine, methionine, alanine, phenylalanine, tyrosine, tryptophan, arginine, lysine, glycine, histidine, aspartate, asparagine, glutamate, glutamine, proline and cysteine. Alternatively, the amino acid residue corresponding to L266 in wild type LHK1 histidine kinase (Xaa) is conservatively substituted with an amino acid selected from among phenylalanine, tyrosine, tryptophan, histidine or proline. Said latter group may be extended to include either of the 3 following groups of amino acids (arginine, lysine, aspartate, glutamate) or (aspargine, glutamine, serine, threonine) or (cysteine, methionine, isoleucine, valine, glycine, alanine). This mutant histidine kinase of the invention includes homologues from a number of plants in which the amino acid residue corresponding to L266 in wild type LHK1 histidine kinase (based on alignment as shown in Table 2) is deleted or substituted with any amino acid other than leucine. More specifically the invention includes mutant histidine kinase homologues from Lotus japonicus (mutant form of Lhk1; SEQ ID NO:6, 15), M. truncatula (mutant form of ABE94286; SEQ ID NO:16), Arabidopsis (mutant form of BAB33311; SEQ ID NO:9, 17), rice (mutant form of XP_{—}469566; SEQ ID NO:18), maize (mutant form of BAE80688; SEQ ID NO:12, 19) and Cucurbita maxima (mutant form of CAF31355.1; SEQ ID NO:14, 20) or orthologues or allelelic variants thereof, wherein Xaa is any amino acid other than leucine. In one embodiment the mutant histidine kinase is one in which Xaa is selected from the group phenylalanine, tyrosine, tryptophan, histidine or proline, or alternatively Xaa is phenylalanine. The orthologue or allelic variant of the mutant histidine kinase of the invention shares a percent sequence identity with said mutant histidine kinase, selected from the group consisting of at least 60, 65, 70, 75, 80, 85, 90, 95, and 98 percent, or truncation thereof, wherein said kinase has all of the functional properties of the mutant histidine kinase of the invention, such that when expressed in a plant of the invention it confers the ability to form spontaneous nodules. In a further embodiment the orthologue/variant allele is a mutant histidine kinase that is a cytokinin receptor protein that is cytokinin independent.

This single amino acid substitution or deletion encoded by the mutant snf2 allele of the Lhk1 gene or its homologues is sufficient to confer a spontaneous nodulation phenotype on the roots of transformed wild-type plants expressing a transgene comprising the gene corresponding to the mutant snf allele (Table 1 and FIG. 3). The snf2 allele acts as a dominant gain-of-function gene, since spontaneous nodulation is seen in a wild type genetic background, and is absent in plants transformed with the wild type Lhk1 gene. Hence, expression of a single copy of the mutant histidine kinase of the invention in a plant, as in the case of a heterozygous plant, is sufficient to confer the snf2 spontaneous nodulation phentoype.

The present invention further provides a DNA molecule encoding said mutant histidine kinase homologue (wherein Xaa is phe) from Lotus japonicus (SEQ ID NO:4, 5), M. truncatula (mutant form of Accession AC141922.19; SEQ ID NO:7), Arabidopsis (mutant form of AB049935.1; SEQ ID NO:8), rice (mutant form of NT_{—}079916.2; SEQ ID NO:10), maize (mutant form of AB206392.1; SEQ ID NO:11) and Cucurbita maxima (mutant form of AJ628045.1; SEQ ID NO:13), or fragments thereof encoding a full-length or truncated functional mutant histidine kinase capable of causing spontaneous nodulation when expressed in a plant.

According to the present invention the DNA sequence encoding the mutant histidine kinase protein of the invention is operably linked to a promoter DNA sequence capable of driving expression of said histidine kinase in a plant, and to a 3′ terminator sequence. The promoter can be a promoter directing expression of said histidine kinase in root tissues of a plant and/or in cells destined to become nodule primordia and mature into nodules. Suitable examples of a promoter and terminator include the promoter and terminator sequence of the corresponding wild type Lhk1 gene. In one embodiment of the invention, the promoter used to direct expression of the mutant histidine kinase is a regulated (e.g. tissue or cell-type specific promoter) which includes the native promoter of the Lhk1 gene or its homologues as defined in the present invention. An example of a heterologous constitutive promoter includes the 35SCaMV promoter (Acc.No:V00141, J02048). A transgene (gene cassette) comprising a DNA sequence encoding a mutant histidine kinase of the invention operably fused to a promoter sequence and optionally a terminator sequence can be constructed by recombinant DNA techniques.

According to the present invention, a transgene comprising a DNA sequence encoding the mutant histidine kinase can be used to generate a plant expressing the mutant histidine kinase of the invention. The transgene can be stably integrated into the genome of a host plant by transformation techniques well know to one skilled in the art. Furthermore, binary vectors and Agrobacterium tumefaciens-based methods for the stable integration of transgenes into all major cereal plants are know, as described for example for rice (Hiei et al, 1994, The Plant J. 6; 271-282), and maize (Yuji et al, 1996, Nature Biotech. 14: 745-750). A DNA sequence encoding a mutant histidine kinase can also be introgressed into another plant by crossing with a genetically modified plant expressing the mutant histidine kinase of the invention.

The genetically modified plant of the invention, whether generated by mutagenesis, transformation with a transgene of the invention, or introgression of said transgene, can be used in a breeding program, in order to select plants with the ability to fix nitrogen, or enhanced nitrogen fixation ability, that have inherited the gene encoding the mutant histidine kinase. The invention thus encompasses a genetically modified plant, produced by transformation of a natural plant, that is capable of spontaneous nodulation. The expression of a gene encoding a mutant histidine kinase in a nitrogen-fixing plant, such as a member of the Leguminoseae (such as soybean, bean, pea, peanut, chickpea, cowpea, lentil, pigeonpea, alfalfa and clover), has particularly utility with respect to enhancing the nitrogen-fixing ability of said plant under one or more environmental growth conditions. The expression of a gene encoding a mutant histidine kinase in a crop plant that does not naturally fix nitrogen, such as a dicotyledenous plant or a monocotyledenous plant including a member of the cereals (such as wheat, rye, oats, barley, sorghum, millet, maize, Poaceae grass and rice), has particularly utility with respect to conferring the ability to fix nitrogen. Plants, as well as plant progeny, selected in such a breeding program may be cultivated for the purpose of harvesting a crop, where the crop may be the vegetative plant parts, e.g. leaf, stem or tuber, or reproductive parts, including flowers, seed, caryopsis, cob or fruit.

The examples given below serve to illustrate the various embodiments of the invention and their respective features. They demonstrate that a plant, e.g. Lotus, that is homozygous or heterozygous for a gene encoding the mutant histidine kinase polypeptide of the invention, forms spontaneous nodules that can be infected by nitrogen-fixing symbiotic bacteria e.g. M. loti, and which are capable of fixing nitrogen and supporting plant growth under nitrogen-limiting conditions. The formation of nitrogen-fixing nodules following inoculation with nitrogen-fixing bacteria in said spontaneously nodulating plants is not dependent on nod factor production by the infecting bacteria or nod factor perception by the infected plant. This indicates that only a subset of nodulation-related genes are required in order for nitrogen fixation to occur in a spontaneously nodulating plant expressing a mutant histidine kinase following inoculation with a rhizobium bacterium. The unique properties of the mutant histidine kinase of the invention may be exploited both to enhance nitrogen fixation in existing nitrogen fixing plants, as well as in establishing nitrogen fixation in non-nodulating plants.

### Example 1

### Positional Cloning and Identification of the Mutant snf-2 Gene in Lotus japonicus

Lotus mutants (snf2-1 and snf2-2) having a spontaneous nodulation phenotype, originates from an EMS screen of Lotus japonicus ecotype Gifu seeds. The mutant snf-2 gene in Lotus japonicus, giving rise to spontaneous nodulation, has been localized on the long arm of chromosome IV, approximately 1 cM from the end, at a locus named Lhk1.

The location of the snf2 gene was determined by fine mapping using microsatellite markers (TM markers) and single nucleotide polymorphic markers developed from BAC or TAC clones anchored to the genetic map of the Lhk1 region. Mapping was performed on an F2 population established from a cross between Lotus japonicus ecotypes Miyakojima and MG-20. The fine map was used to build a physical TAC/BAC contig comprising six BAC/TAC clones from MG20 that were assembled to cover the Lhk1 region between the two flanking markers TM1146 and TM0069 (FIG. 2). Since snf2 is a dominant mutation, wild-type F2 plants, rather than mutant plants, were scored for mapping purposes. Genotyping of 853 wild-type plants that did not develop root nodules spontaneously was used to identify markers delimiting the locus, corresponding to the snf2 mutation, to a region of 120 kb. Fifteen genes were predicted within this region, one of which was predicted to encode a cytokinin receptor. The sequence of both the wild type [SEQ ID NO:1] and mutant snf2 alleles [SEQ ID NO:4] of this candidate gene were determined, which revealed a C to T nucleotide substitution in both snf2 alleles. The wild type gene was designated Lotus histidine kinase (Lhk1) gene encoding a LHK1 polypeptide [SEQ ID NO:3]. The single nucleotide transition (C to T) in an exon sequence of the mutant snf2 allele, encodes a polypeptide having a phenylalanine (F266 in SEQ ID NO:6) in substitution of the conserved leucine (L266 in SEQ ID NO:3) in the polypeptide encoded by the wild type gene, and identifies snf2 as an allele of a Lotus histidine kinase (Lhk1) gene.

### Example 2

### Cloning and Identification of the Lhk1 cDNA Corresponding to the Transcript of the Wild Type Lhk1 Gene in Lotus japonicus

A full-length Lhk1 cDNA (3568 bp) was isolated from a λ ZAPII cDNA library prepared from mRNA isolated from M. loti inoculated Lotus japonicus roots. The Lhk1 cDNA was sequenced [SEQ ID NO:2], from which the transcription start site was determined to lie at least 137 nucleotides upstream of the start codon and the coding sequence was followed by a 3′ untranslated region of approximately 445 nucleotides. Alignment of genomic and cDNA sequences defined a primary structure of Lhk1 consisting of 11 exons (FIG. 2B). The nucleotide sequence of the mutant snf2 allele of the Lhk1 cDNA is designated SEQ ID NO:5.

### Example 3

### Expression of the snf2 Allele in Wild Type Lotus Roots Confers a Spontaneous Nodulation Phenotype

Transgenic expression of the wild type or mutant snf2 allele in wild type Lotus roots was performed in order to confirm in planta the genetic basis for the spontaneous nodulation phenotype. A genomic fragment of 12.7 kb from the BAC clone 1 K18 was used to clone out the wild type Lhk1 gene including a 2.2 kb promoter region, and its Snf2 allele comprising the C→T transition, which were then sub-cloned into the pIV10 plasmid. The constructs were integrated into A. rhizogenes strain AR12 by tri-parental mating. Transformation was performed as described by Stougaard (1995) Method Mol Biology 49: Plant Gene Transfer and Expression Protocols, p. 49-63, and included transformation with an empty vector control. Root nodulation, scored in the absence of rhizobial induction, was only detected in roots transformed with the snf2 allele, while the wild type Lhk1 gene failed to confer spontaneous nodulation on wild type roots (table 1, FIG. 3).

TABLE 1
Induction of spontaneous nodules on wild type hairy roots transformed
with wild type Lhk1 gene or its snf2 gain-of-function allele

| Transformation | % spontaneous | nodule number/ | nodule number/ |
|---|---|---|---|
| construct | nodulation* | plant* | nodulated plant* |
| empty vector | 0 (0/15) | 0 | 0 |
| Wild type Lhk1 | 0 (0/41) | 0 | 0 |
| allele | | | |
| snf2 allele | 37 (30/81) | 0.87 | 2.36 |

*Transgenic roots were scored in absence of M. loti.

The ability of the snf2 allele to confer spontaneous nodulation in transformed roots having a wild-type genetic background serves to confirm the dominant nature of the snf2 allele. Furthermore it is surprisingly shown that a cytokinin independent histidine kinase (snf2), when under the control of a regulated promoter e.g. Lhk1 gene promoter, confers the controlled organogenesis of root nodules, as against uncontrolled cell proliferation leading to massive nodule development. Controlled formation of spontaneous nodules, capable of infection and nitrogen fixation in plants transformed with the snf2 allele, enhances their capacity to fix nitrogen during cultivation. Since the only structural difference between the wild type Lhk1 gene and its snf2 allele lies in a point mutation encoding a single amino acid substitution, these data demonstrate that spontaneous nodulation is the result of an L/F266 substitution in a LHK1 polypeptide.

### Example 3

### Lotus LHK1 is a Member of the Cytokinin Receptor Family

Annotation of the Lhk1 cDNA clone reveals an open reading frame of 2979 nucleotides that is predicted to encode a cytokinin receptor protein (LHK1) consisting of 993 amino acids with a predicted mass of 110 kD (FIG. 4). At the N-terminus, two membrane spanning segments are located between amino acids 37 and 57 and between amino acids 328 and 357. Located between these segments are motifs characteristic for the cyclases/histidine kinases associated sensory extracellular (CHASE) domain. This predicted extracellular domain is followed by a putative intracellular histidine kinase between amino acids 379 and 693 and a receiver domain between amino acids 852 and 985. These domains are characteristic for two-component regulatory systems operating through a phospho-relay.

Comparative analysis defines the Lotus LHK1 protein as a member of the cytokinin receptor family which includes proteins from Medicago truncatula, Arabidopsis, rice and maize (table 2). The Lotus LHK1 protein shares an amino acid sequence identity of 83%, 68%, 58% and 49% respectively, with protein homologues from M. truncatula (ABE94286), Arabidopsis (BAB33311), rice (XP_{—}469566) and maize (BAD01584). Among the three Arabidopsis cytokinin receptors, LHK1 is most similar to AHK4/(Cre1) that is important for normal root development and serves a function in perception of externally supplied cytokinin (Mahonen et al. (2000), Genes Dev. 4: 2938). The leucine 266 substituted by a phenylalanine 266 in the snf2 allele, is localised in a conserved motif shared among the extracellular CHASE domains of histidine kinase receptors (FIG. 4).

TABLE 2
Alignment of Histidine kinases from Lotus, Arabidopsis, maize, rice and Medicago.

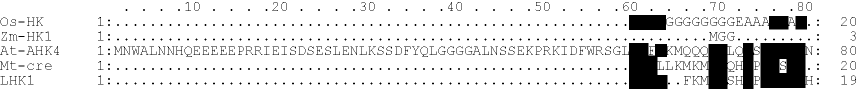

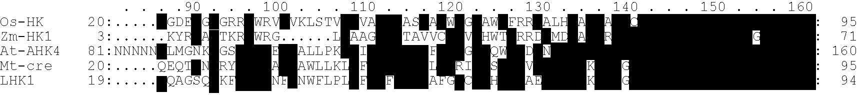

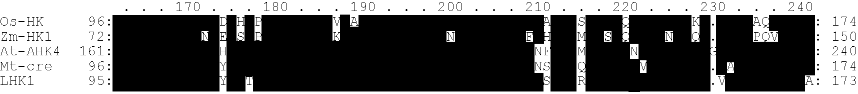

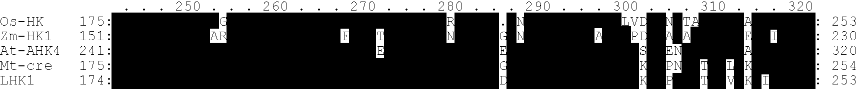

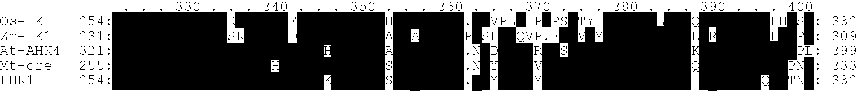

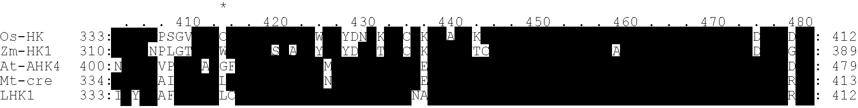

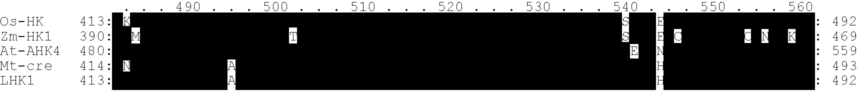

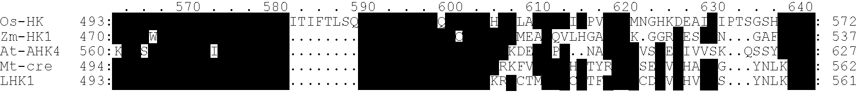

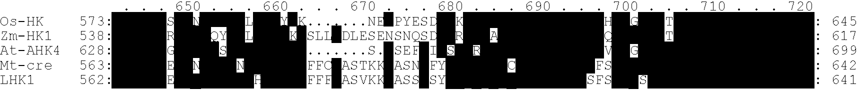

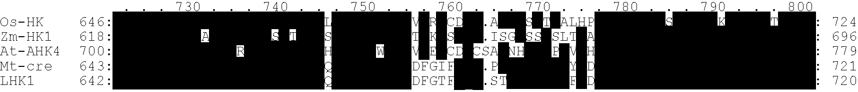

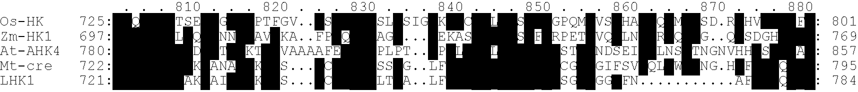

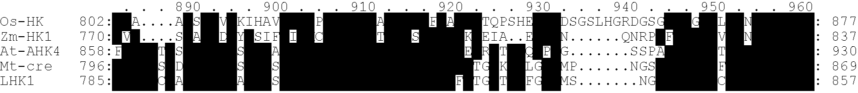

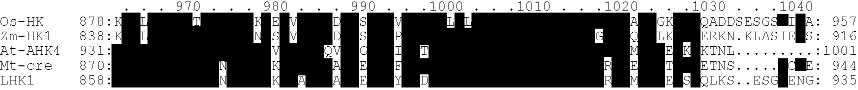

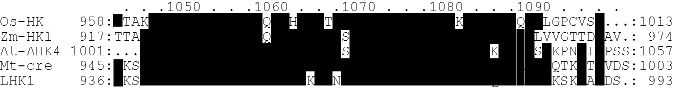

*Indicates the amino acid substitution at position 266 Leucme to Phenylalanine in the mutant

### Example 4

### Cytokinin Independent Activity

The spontaneous nodulation phenotype conferred by the snf2 allele is shown to be due to a cytokinin-independent function of the mutant LHK1 cytokinin receptor encoded by this allele, which is characterised by a L/F266 substitution in the CHASE domain (FIG. 4). The in vivo activity of Lotus wild type and mutant LHK1 cytokinin receptors and their cytokinin response was demonstrated by means of a two-component phosphorelay assay developed in E. coli (Suzuki et al., (2001) Plant Cell Physiol. 42: 107). In this assay cytokinin induction can be determined without interference from endogenous cytokinin levels, cytokinin penetration and interfering metabolic pathways present in plants. The phosphorelay exploited in this assay is normally involved in regulating bacterial extracellular polysaccharide synthesis through activation of the cps operon in response to a sensor (RcsC), which has a domain structure similar to plant cytokinin receptors. Functional expression of a cytokinin receptor in an E. coli strain deleted of RcsC sensor allows cytokinin perception to be read out as β-galactosidase activity from the expression of a cps::lacZ fusion.

Accordingly the Lhk1 cDNAs, corresponding to wild type and snf2 allele were cloned into the pIN-III expression vector and transformed into the sensor-negative SRC122 (ΔRcsC) strain harbouring the cps::lacZ gene. The transformants were grown overnight in liquid Luria-agar and then spotted on LB plates, supplemented with and without t-zeatin, kinetin, BAP (200 μM) each and thidiazuron (50 μM). The LB media was further supplemented with X-Gal (5-bromo-4-chloro-3-indolyl-β-D-galactosidase), a β-galactosidase substrate. After 40 hours incubation at room temperature (25° C.), the plates were photographed. E coli strain (SRC122) was taken as a negative control. Expression of the snf2 allele (mutant LHK1 cytokinin receptor) is seen to induce β-galactosidase activity in absence of cytokinin (FIG. 5A). In contrast, wild type LHK1 induced β-galactosidase activity in a cytokinin-dependent fashion (FIG. 5A). The level of β-galactosidase activity induced by expression of the wild type and snf2 alleles was quantitatively determined in the transformants after overnight growth at 37° C. in LB medium according to the following protocol. Overnight cultures were diluted 1:1000 in 50 ml LB and further grown for 24 hours at 37° C. After the incubation period, 1 ml of culture was centrifuged for 10 min at 13000 rpm. The OD₆₀₀ of the remaining culture was measured. The pellet was resuspended into 1 ml 0.85% NaCl. 200 μl of the suspension were adjusted to 1 ml with Z buffer (Guarentee (1983) Methods Enzymol., 101: 183). Two drops 0.1% SDS and 1 drop chloroform were added. The samples were vortexed and left at 30° C. for 5 min. 200 μl of o-Nitrophenyl-β-D-galactopyranoside ONPG (4 mg/ml) were then added to the sample, which was incubated further at 30° C. When the samples had obtained an appropriate yellow colour (corresponding to OD₄₂₀=0.300−0.600), the reaction is stopped by adding 500 μl of 1M Na₂CO₃. The stop time was scored. The OD₄₂₀ was measured within 1 hour by spectrophotometry. The β-galactosidase activity was calculated as follow: Relative units=(0D₄₂₀*1000)/(OD₆₀₀*min*ml) (Guarentee, (1983) supra).

As shown in FIG. 5B, expression of the snf2 allele of LHK1 in E. coli cells induced a three fold higher level of β-galactosidase activity than control, non-transformed E. coli cells and cells expressing wild type LHK1. Cytokinin addition results in a two fold induction of β-galactosidase activity in LHK1 expressing cells while snf2 allele expressing cells respond with only a marginal increase in activity. These results demonstrate that LHK1 is a cytokinin receptor, while the mutant receptor encoded by the snf2 allele is constitutively active, whose activity is comparable to the cytokinin-induced activity of the wild type receptor. While not wishing to be bound by theory, it is proposed that the extracellular CHASE domain, that normally binds cytokinin to activate the kinase (Kakimoto, (2001) Plant Cell Physiol. 42: 677; Anantharaman and Aravind, (2001)Trends Biochem Sci. 26: 579; Pas et al., (2004), FEBS Lett. 576: 287) is locked within an active conformation in the mutant (snf2) receptor. These properties of the mutant LHK1 would explain both the genetic dominant nature of the snf2 allele and the phosphorelay assay results.

### Example 5

### Impact of the snf2 Allele on Regulation of Cell Growth and Differentiation

The spontaneous conversion of root cortical cells into root nodule initials seen in snf2 mutants is probably due to constitutive activity in one or more steps of the pathway controlling cell differentiation. The in vitro tissue culture performance of snf2 cells provides a means to assess the global impact of the snf2 allele on cell differentiation processes and/or interference with normal phytohormone responses. Accordingly, hypocotyl and root explants (FIGS. 1 and 6) from 10 days old wild type and snf2 plants (cultivated on ½ B5 salt medium) were grown on 36 ratios of naphtalene acetic acid (NAA) and 6-benzylaminopurine (BAP) for three weeks. The range of cytokinin and auxin concentrations employed included levels normally used for optimal in vitro culture of wild type explants. The explants were moved to a fresh media every week. Representative explants for each hormone combination were arranged on a Petri dish and photographed.

As shown in FIG. 1G, H and FIG. 6, there is little observable difference in callus growth from hypocototyl or root explants of plants with wild type and snf2 alleles. Overall, the hormone dose response is similar. Only at high auxin concentrations (2 μg/ml NAA) did snf2 explants survive slightly better than wild type explants. Conversely, on cytokinin (BAP) in absence of auxin, snf2 explants developed less callus than wild type. When cultivated on tissue-culture plates without any phytohormones, green callus grew from the basal end of snf2 root segments, in a manner reminiscent of a cytokinin-induced tissue response, and clearly different from wild type root segments (FIGS. 1I and J). Co-segregation of this green callus phenotype and the snf2 allele shows that this un-induced root callus formation was caused by the gain-of-function cytokinin receptor encoded by the snf2 allele.

Microscopy on thin sections of wild-type and snf2 roots (FIGS. 1 C,D and E,F) show at least one extra cell layer in the xylem vessels of snf2 roots. After 6 days of incubation on hormone-free media, additional pericycle cell layers originating from periclinal divisions were observed together with an increase in cell number of the snf2 root vasculature while no changes were observed in wild type roots (FIG. 1E, F).

### Example 6

### Expression of Lhk1 Gene in Organs and Lhk1, Lrr5 and Nin Genes in Wild Type and snf2 Lotus Plants in Response to Cytokinin

Steady state levels of Lhk1 and ARR transcripts were determined in roots and other plant organs by quantitative RT-PCR analysis according to the following protocol.

Wild type and snf2 seeds were surface sterilised and germinated as described previously (Handberg, and Stougaard, (1992) Plant J. 2: 487). Plants were grown with 16 h light/8 h dark period at 21° C. Total RNA was isolated from roots, nodules, leaves, flowers and pods of inoculated wild type L. japonicus. mRNA was extracted from roots of wild type and snf2 plants grown for 2 weeks on ¼ B&D (Broughton and Dilworth, (1971) Biochem. J. 125: 1075) supplemented with 0.5 μM potassium nitrate. Roots were treated with 10 μM BAP for three time points, 30 minutes, 3 hours and 8 hours. Untreated roots were taken as control. mRNA was also extracted from root segments of wild type and snf2 plants grown for 10 days on hormone-free B5 salt medium and B5 salt medium supplemented with 0.5 μg/ml of BAP. Dynabeads mRNA direct kit (Invitrogen) was used for the extraction of mRNA.

First strand cDNA was prepared using reverse transcriptase (Fermentas). Quantitative PCR was performed on a Light Cycler (Roche Molecular Biochemicals) using Fast Start DNA master SYBR green kit (Roche) to amplify the target transcripts from 5 μl diluted cDNA. Four house keeping genes (Czechowski et al., (2005) Genome analysis 139: 5) were used to determine the relative expression of target genes (see below). For each treatment, normalized relative ratios of the target genes and the four independent house keeping genes have been calculated using the Relative Quantification Software (Quant) from Roche. The geometric mean (Vandesompele et al., (2002)Genome Biology 3:1) of the relative expression ratios for the three biological and technical replicates and the corresponding 95% intervals of confidence have been calculated as described previously (Radutoiu et al., (2003) Nature 425: 585).

The following primer pairs were used for quantitative PCR:

Independent house keeping gene primer sequences:

1. PP2A-TC9878-homolog of AT1G13320-protein
phosphatase 2A
5′-GTAAATGCGTCTAAAGATAGGGTCC-3′
5′-ACTAGACTGTAGTGCTTGAGAGGC-3′
2. UBC-CB828248 homolog of AT5G25760-ubiquitin-
conjugating enzyme
5′-ATGTGCATTTTAAGACAGGG-3′
5′-GAACGTAGAAGATTGCCTGAA-3′
3. TB2C-B1418560-tubulin beta chain
5′-GCTCACCACCCCAAGCTTTGG-3′
5′-TGTCAATGGAGCAAACCCAACC-3′
4. ATP-AW719841-ATP synthase-
5′-AACACCACTCTCGATCATTTCTCTG-3′
5′-CAATGTCGCCAAGGCCCATGGTG-3′
Target gene primer sequences:
Lhk1
5′-AATTTGGTGAACCGAAGGGTCGCCG-3′
5′-TCGACGAGTGGCCTCAAACCCATCC-3′
snf2 allele of Lhk1

| | |
|---|---|
| Snf2LC3Fwd: | 5′-AGAGGTCTTAAAGCCATTGTG-3′ |
| Snf2LC6Rev: | 5′-TATCAGGCTGAAATAATGCCG-3′ |

Nin-AJ239041.
5′-AGGAGCCCAAGTGAGTGCTA-3′
5′-GCCATCAAGGTATATGACGAG-3′
ARR5-CB827384-homolog of AT3G48100-ARR5
5′-TCTTGACTCGAATTGATAGGTGC-3′
5′-GATAGAGATGGCCTGCAACTACTG-3′

Transcript analysis shows Lhk1 to be expressed at the highest level in roots, nodules and leaves, but transcripts were present in all organs tested (FIG. 7A).

Cytokinin-induced changes in cellular processes in plants are accompanied by increased expression of response regulators (ARR) genes belonging to the type-A class (Hutchison and Kieber, (2002) The plant Cell 14: 47). Ten genes encode ARRs in Arabidopsis and their transcripts have been found in all adult tissues. Type-A ARR genes are transcriptionally induced by cytokinin and ARR4 and ARR5 are rapidly induced primary response genes. Since the mutant receptor protein encoded by the snf2 allele showed constitutive activity in the E. coli test system, the activation of a Lotus ARR5 homolog (named Lrr5) was determined in snf2 and wild-type roots (FIG. 7). A two-fold higher level of Lrr5 transcript was found in root explants of snf2 mutants incubated on hormone-free B5 medium compared to wild type explants, while cytokinin [BAP] addition to the B5 medium increased the Lrr5 transcript level two to three-fold in both snf2 and wild-type explants (FIG. 7B). Direct cytokinin treatment of roots also increased the Lrr5 transcript level in snf2 and wild-type roots, but in this experiment, a difference in expression between untreated snf2 and wild type roots was not detected (FIG. 7C).

Lhk1 gene transcripts are also seen to increase rapidly in response to cytokinin treatment in both wild type and snf2 mutants (FIG. 7D), which is consistent with the cytokinin inducibility of its homologue in Arabidopsis (AHK4 gene). The Nin gene in Lotus, which is required for the initiation of nodule primordial (FIG. 5C), is also transcriptionally upregulated by cytokinin (FIG. 7E), but the transcript levels in untreated and treated snf2 roots were not significantly different from that of wild type roots. Ectopic expression of Nin in root explants of snf2 mutants incubated on hormone free B5 medium was not detected (FIG. 2F).

In Arabidopsis, the ARR response pathway is desensitized after prolonged exposure to cytokinin (Rashotte et al., (2003), Plant Physiol. 132: 1998), which may explain the relatively small or lack of upregulation of Lrr5 and Nin in snf2 mutant roots.

### Example 7

### The Growth Habit of Snf2 Lotus Plant is Sensitive to Cytokinin

Although transcriptional changes in the snf2 Lotus plants are limited, plant growth was seen to be strongly affected by application of externally supplied cytokinin. Accordingly, wild type and snf2 plants were grown on ½ B5 salt with and without increasing concentrations of BAP for 3 weeks. Shoot and root length of at least 60 plants for each treatment was measured. In line with the constitutive activity of the mutant cytokinin receptor encoded by the snf2 allele, both shoot and root growth of snf2 plants were hypersensitive to cytokinin compared to wild type (FIG. 8).

### Example 8

### The snf2 Root Phenotype Co-Segregates with the snf2 Mutation

The roots of snf2 plants, grown on hormone-free ½ B5 salt media, are characterised by enhanced cell division in the pericycle and vascular tissue (FIG. 1 D,F). This growth pattern, which leads to a swollen root phenotype, is attributed to the expression of the snf2 allele. To confirm that the root swelling phenotype indeed co-segregates with the spontaneous nodulation phenotype, the snf2-2 mutant allele was backcrossed to the wild type Gifu ecotype and the F2 plants [50] were grown on ¼ B&D media for sufficient time to allow the scoring of spontaneous nodulation phenotype (˜5 weeks). The roots of the nodulating and non-nodulating plants were cultivated for 3 weeks on hormone-free media, and then scored for presence and absence of root swelling. Wild type and snf2 plants were taken as controls. Only roots from F2 plants developing spontaneous nodules showed swelling of the root segments that was comparable to the swelling of the snf2 root segments. This indicates that expression of the cytokinin independent mutant Lhk1 protein in Lotus plants confers both the snf2 root and shoot growth phenotype and the spontaneous nodulation phenotype.

### Example 9

### snf2 and Cytokinin Signalling Acts Downstream of Nod Factor Induced Signal Transduction Pathway

The phenotype of snf2 mutants suggests that cytokinin signalling is part of, or acts downstream, of the Nod-factor induced signal transduction pathway (FIG. 5C). To test this hypothesis, the snf2 gene construct was transformed into mutant plants carrying mutations in genes constituting the common signal transduction pathway shared with mycorrhizal fungi or into mutant plants impaired in upstream or downstream genes.

Accordingly, seven single and double L. japonicus nodulation mutants were transformed using Agrobacterium rhizogenes carrying the snf2 mutant gene construct or an empty vector as described previously (Stougaard, 1995 supra). All the mutants except sym35 were genotyped by sequencing PCR products covering the mutation using the following primers:

| | |
|---|---|
| nfr1 | |
| Nfr1-1 MseI CAPS fw(56) | 5′-CGCTGGTTTACCCATAAACGTGT |
| | TC-3′ |
| Nfr1-1 MseI CAPS rv(55) | 5′-GGGCAAATGCATTTGTGCTGA |
| | G-3′ |
| nfr5 | |
| K2fwB | 5′-CCAGCTAGGTGATAGCTACG-3′ |
| K2revC | 5′-CCAGAAGATGAATGCTGCTT |
| | T-3′ |
| S5R rev5(64) | 5′-GGTATTAGAACGCCCCCTGG-3′ |
| symRK (wild type) | |
| symRK fw1 | 5′-CTGAGTTTGGACCCCTTTTG-3′ |
| symRK rev1 | 5′-ACGCCCTTATGAAAATGTGG-3′ |
| symRK (mutant gene) | |
| Lore2 5′ LTR out P | 5′-GGAGCTCTGATACCAATGTTAG |
| | G-3′ |
| cac41.5F | 5′-CGGCAATAGAGCGCTGGAGAGTT |
| | G-3′ |
| ccamk | |
| LTsym15(60)fw | 5′-TATGACACAGATAGATCAGG |
| | G-3′ |
| LTsym15(60)rev | 5′-GAGAGCGGCTCAATGAATGT-3′ |
| Nin | |
| Reso fwd | 5′-CTCAGAGCACGCTTCTTGGA-3′ |
| Reso rev | 5′-ATCATGTGTGCAATCCATGAT |
| | G-3′ |
| haR1 | |
| #3fw2- | 5′-CCTGAAATGCCTATTCGTTGA |
| | G-3′ |
| #3rev2 | 5′-CACAGCTTCTTCTGCATGCG-3′ |
| snf1 | |
| Ca2fwd | 5′-TGGCTTGCATCCAAACGGC-3′ |
| Ca3-3rev | 5′-ACTATTGTTGTCTCACTTTAGT |
| | G-3′ |
| snf2 | |
| C3fwd | 5′-TGGGATAATTGGTTGCTTGAC |
| | A-3′ |
| C3Brev | 5′-TGACAATGTGAGTTCCAGCA |
| | G-3′ |

Transgenic hairy roots were then monitored for spontaneous nodulation in the absence of rhizobia.

TABLE 3
Spontaneous nodulation in non-nodulating Lotus
mutants transformed with the snf2 gene.

| Mutant* | % spontaneous nodulation | |
|---|---|---|
| nfr1 | 43 | (3/7) |
| nfr5 | 41 | (24/58) |
| nfr1nfr5 | 28 | (33/118) |
| symrk | 36 | (31/87) |
| ccamk | 19 | (18/94) |
| nin | 0 | (0/112) |
| sym35 | 0 | (0/136) |

*Hairy roots of transformed mutants were scored in the absence of M. loti. Roots of control mutant plants transformed with an empty vector did not develop spontaneous nodules.

Spontaneous nodulation observed in nfr1-1, nfr5-2 and nfr1-1 nfr5-2 Nod-factor receptor single and double mutants lacking the earliest electrophysiological responses to Nod-factor (Radutoiu et al., (2003) supra; Madsen et al., (2003) supra), demonstrates that Lhk1 functions downstream of Nod-factor signal perception. The common pathway symRK mutants lacking Ca²⁺ spiking (Stracke et al., (2002) Nature, 417: 959; Niwa et al., (2001) Mol Plant Microbe Interact 14: 848) and the ccamk mutants suggested to be unable to interpret Ca²⁺ spiking (Tirichine et al., (2006) Nature 441: 1153), also develop spontaneous root nodules in transgenic roots transformed with the snf2 gene construct. In the nin and sym35 mutants that are arrested before initiation of cell division induced through the common pathway, no spontaneous nodules were observed in transgenic roots showing that cytokinin signal perception acts upstream of cell division initiation, or operates in a parallel pathway (FIG. 5C). Further evidence for a central role of cytokinin and cytokinin perception in the Nod-factor induced signal transduction amplified through the common pathway comes from the additive effect of snf1-1 and snf2 mutations in double mutants. The snf1-1 mutants synthesize a CCaMK protein impaired in autophosphorylation (Tirichine et al., (2006) supra) and develop an average of seven spontaneous nodules. snf2 mutants develop an average of three spontaneous nodules, while snf1-1 snf2 double mutants exceed both and develop approximately seventeen spontaneous nodules. Conversion of cortical cells into nodule stem cells or the subsequent organ development seems therefore tightly controlled. This theory was tested by crossing the snf2 allele into a hypernodulating har1-1 genetic background (Krusell et al., (2002) Nature 420: 422). In absence of rhizobia, homozygous snf2 har1-1 double mutants developed an average of fourteen spontaneous nodules, while snf2 mutants developed an average of three, and har1-1 none (FIG. 9). These results indicate that only a few cells de-differentiate or that only few de-differentiated cells sustain cell divisions during the snf2 nodule initiation process. The shoot controlled autoregulation of the root nodule number (Krusell et al., (2002) Nature 420: 422) is thus acting downstream of cytokinin signalling and cytokinin induced activation of root nodule founder cells (FIG. 5C). The data clearly demonstrate that cytokinin signalling is necessary and sufficient for the de-differentiation and cell proliferation leading to root nodule formation and that this developmental process can be triggered independently of cytokinin by expression of the dominant snf2 allele. Surprisingly, the phenotype conferred by expression of the snf2 allele, expressed under the control of a regulated promoter (e.g. Lhk1 gene promoter) is characterised by controlled organogenesis of root nodules, rather than uncontrolled, cytokinin-independent, cell proliferation.

### Example 10

### Detection Kit to Distinguish Snf2 Mutant Allele from Wild Type Allele Encoding LKH1 Histidine Kinase

A kit for the detection of the snf2 mutant allele useful for genotype screening purposes comprises at least:

Two alternative sets of dCAPS (derived cleaved amplified polymorphic sequence) primers are provided for detection of wild type versus mutation:

Set one:
Snf2 XmnI/Asp700 dCAPS fw (74)
5′-CACCAAAATTGCTTGGTTACCAGCAAGTTGACCGA
Snf2 XmnI/Asp700 dCAPS rv(69)
5′-CCCTTCATGTGGCCCTTACCCAAC

The primers are suitable for use in a PCR test performed with genomic DNA as the template, where amplification is performed at 60° C. with 30 seconds elongation time and 35-40 cycles. The 225 bp product is cut by Xmnl/Asp700 in the mutant, and not cut in wildtype. The cleaved PCR products can be identified following their separation on a 1% agarose gel.

Set two:
Snf2 RsaI dCAPS fw(72)
5′-GATCCTTTGATGTTGAGTCCCTTGTGGAGAATGTA
Snf2 RsaI dCAPS rv(62)
5′-CTGAAACTCAGAAAATGTACTACAAC

The primers are suitable for use in a PCR test performed with genomic DNA as the template, where amplification is performed at 48° C. with 30 seconds elongation time and 35-40 cycles. The 253 bp product is cut bu Rsal in the wildtype, but uncut in mutant.

The cleaved PCR products can be identified following their separation on a 1% agarose gel.

### Example 11

### Amplification Primers for Cloning the snf2 cDNA Coding Sequence

Primers to amplify the coding sequence from start to stop (cDNA) are:

| | |
|---|---|
| Snf2 cDNA fw | ATGGGTCTTGGGTTCAAGATGCA |
| Snf2 cDNA rev | TCATGAGTCTGAAGCAGGCTTGG |

PCR was performed with annealing at 58° C. and 3 minute 20 seconds elongation time for 25 cycles with cDNA clone as template.

1. A DNA molecule encoding a mutant histidine kinase polypeptide comprising an amino acid sequence selected from the group consisting of:
(a) SEQ ID NO: 6, 15, 16, 17, 18, 19 and 20, wherein the amino acid residue 266 in each of SEQ ID NO: 6, 15, 17, and 18, and amino acid residue 267 in each of SEQ ID NO: 16 and 19, and amino acid residue 261 in SEQ ID NO: 20 is selected from the group consisting of isoleucine, serine, threonine, valine, methionine, alanine, phenylalanine, tyrosine, tryptophan, arginine, lysine, glycine, histidine, aspartate, asparagine, glutamate, glutamine, proline and cysteine,
(b) an amino acid sequence having at least 70% sequence identity with SEQ ID NO: 6, 15, 16, 17, 18, 19, and 20 wherein the amino acid residue 266 in each of SEQ ID NO: 6, 15, 17, and 18 and amino acid residue 267 in each of SEQ ID NO: 16 and 19 is selected from the group consisting of isoleucine, serine, threonine, valine, methionine, alanine, phenylalanine, tyrosine, tryptophan, arginine, lysine, glycine, histidine, aspartate, asparagine, glutamate, glutamine, proline and cysteine, and
(c) a truncation of (a),
wherein said polypeptide is capable of inducing spontaneous nodule formation when expressed in a plant.

2. A DNA molecule according to claim 1, wherein the amino acid residue 266 in each of SEQ ID NO: 6, 15, 17, and 18, and amino acid residue 267 in each of SEQ ID NO: 16 and 19, and amino acid residue 261 in SEQ ID NO: 20 is selected from the group consisting of phenylalanine, tyrosine, tryptophan, histidine and proline.

3. A mutant histidine kinase polypeptide encoded by the DNA molecule of claim 1.

4. A mutant histidine kinase polypeptide according to claim 3, wherein the polypeptide is cytokinin independent.

5. A genetically modified plant characterised by a having a nucleotide sequence encoding a mutant histidine kinase polypeptide comprising an amino acid sequence selected from the group consisting of:
(a) SEQ ID NO: 6, 15, 16, 17, 18, 19 and 20, wherein the amino acid residue 266 in each of SEQ ID NO: 6, 15, 17, and 18, and amino acid residue 267 in each of SEQ ID NO: 16 and 19, and amino acid residue 261 in SEQ ID NO: 20 is selected from the group consisting of isoleucine, serine, threonine, valine, methionine, alanine, phenylalanine, tyrosine, tryptophan, arginine, lysine, glycine, histidine, aspartate, asparagine, glutamate, glutamine, proline and cysteine,
(b) an amino acid sequence having at least 70 percent sequence identity with SEQ ID NO: 6, 15, 16, 17, 18, 19 and 20, wherein the amino acid residue corresponding to 266 in each of SEQ ID NO: 6, 15, 17, and 18, and amino acid residue 267 in each of SEQ ID NO: 16 and 19, and amino acid residue 261 in SEQ ID NO: 20 is any amino acid other than leucine, and
(c) a truncation of (a),
wherein said plant is capable of spontaneous nodule formation.

6. A genetically modified plant according to claim 5, wherein the mutant histidine kinase polypeptide is cytokinin independent.

7. A genetically modified plant according to claim 5, wherein the amino acid residue 266 in each of SEQ ID NO: 6, 15, 17, and 18, and amino acid residue 267 in each of SEQ ID NO: 16 and 19, and amino acid residue 261 in SEQ ID NO: 20 is selected from the group consisting of phenylalanine, tyrosine, tryptophan, histidine and proline.

8. A genetically modified plant according to claim 5, wherein the mutant histidine kinase polypeptide is encoded by a nucleic acid molecule having a nucleic acid sequence selected from the group: SEQ ID NO: 4, 5 and 7, or the coding sequence thereof.

9-11. (canceled)

12. A genetically modified plant according to claim 5, further comprising a homologous promoter nucleotide sequence operably linked to the nucleotide sequence encoding the polypeptide.

13. A genetically modified plant according to claim 5, further comprising a heterologous or homologous promoter nucleotide sequence operably linked to the nucleotide sequence encoding the polypeptide.

14. A genetically modified plant according to claim 13, wherein said promoter is a homologous or heterologous regulated promoter.

15. A genetically modified plant according to claim 5, wherein said plant is a monocotyledonous plant.

16. A genetically modified plant according to claim 5, wherein said plant is a dicotyledonous plant.

17. A genetically modified plant according to claim 15, wherein said plant is selected from among the group consisting of rice, barley, maize, oats, rye, sorghum, wheat and Poaceae grass.

18. (canceled)

19. A method of producing a genetically modified plant of claim 5, characterised by introducing a gene cassette comprising said nucleotide sequence encoding said polypeptide into the plant and selecting a transgenic plant and its progeny expressing said polypeptide.

20. A method according to claim 19, wherein the gene cassette is introduced into the plant by transformation.

21. A method according to claim 19, wherein the gene cassette is introgressed introduced into a the plant by sexual crossing with the transgenic plant comprising said gene cassette.

22. (canceled)

23. Seed of the genetically modified plant according to claim 5.

24. A crop comprising a genetically modified plant according to claim 5.

25. (canceled)

26. A plant selected by the method of claim 28.

27. A plant selected in the breeding program of claim 26, wherein the mutant histidine kinase polypeptide is cytokinin independent.

28. A method of selecting the genetically modified plant according to claim 5, comprising:
1) breeding plants having a nucleotide sequence encoding a mutant histidine kinase polypeptide comprising an amino acid sequence selected from the group consisting of:
(a) SEQ ID NO: 6, 15, 16, 17, 18, 19 and 20, wherein the amino acid residue 266 in each of SEQ ID NO: 6, 15, 17, and 18, and amino acid residue 267 in SEQ ID NO: 16 and 19, and amino acid residue 261 in SEQ ID NO: 20 is selected from the group consisting of isoleucine, serine, threonine, valine, methionine, alanine, phenylalanine, tyrosine, tryptophan, arginine, lysine, glycine, histidine, aspartate, asparagine, glutamate, glutamine, proline and cysteine,
(b) an amino acid sequence having at least 70% sequence identity with SEQ ID NO: 6, 15, 16, 17, 18, 19 and 20, wherein the amino acid residue corresponding to 266 in each of SEQ ID NO: 6, 15, 17, and 18, and amino acid residue 267 in SEQ ID NO: 16 and 19, and amino acid residue 261 in SEQ ID NO: 20 is any amino acid other than leucine, and
(c) a truncation of (a), and
2) selecting from among the bred plants, plants encoding the mutant histidine kinase polypeptide and being capable of spontaneous nodule formation.
